SEQUENCE LISTING (3 sequences)

SEQ ID NO: 1 - DNA, 3945 bp, Canis familiaris
gcacttggctcagaggatcctgtcccagggtgatggcatttagccagtggatgccaggtgcccaggctgagccagcgtccccggcgaggagcccaaaggctgctgtgcgagagaagaggcgcggcggagccctgtaaatttgttgccaagaagaggaacaatgaattcctcatttcacttgcttttcttggatctcaactcgaatgccacagagggcaacttttcaggaccaaatgtcaagaacaagtcttcgccgtgtgaagagatgggcattgctgtggaggtgtttctgactctaggtctcatcagcctcttggagaacatcctggtcataggagccatagtgaagaacaagaacctgcactcccccatgtatttctttgtgtgcagcttagcggtagctgacatgctagtgagcatgtctaacacctgggagaccatcaccatatacttaatatataataagcacgtggtgatagcagatgcttttgtgcgtcacattgacaacgtgttcgactccatgatctgcatttccgtggtggcttccatgtgcagcttgctggccatcgcggtggacaggtacgtcaccatcttctacgccctgcgctaccaccacatcatgaccgtgaggcgttccagagtgattatcacgtgtatctggaccttttgcacaggctgcggcatcgttttcatcgtctactacgagtccacgtatgtcatcgtttgcctcatctccatgttcttcaccatgctcttcctcatggtgtccctgtacatacacatgttcctcctggcgcggactcacgtcaagcggatagcagctctgcgtggctgcagctctgtgaggcagacggccagcatgagaggggccgtcaccctgaccatgttgctgggcatcttcatcgtgtgctgggctccgttcttcctccatctcatcttgatgatttcctgcccccagaacttctactgttcttgctttatgtcttactttaatatgtacctcatactcatcatgtgtaattctgtcatcgaccctctgatatatgccttccgcagccaggagatgaggaagtccttcaaagagatcatttgttgccacgctttccgaataccccgtaggttccttagcaggtattaagcaagaagttcttccttatgcaccttggtacagccagggagggagccagaaagctggaaatcagaagcacgcatcctgtttatcttttttccatgtcgaagttgtcctgtatttccccctgcggaagttgttgggtcggacgagcacgtgccgcttacccatttggggaacggtgtgtgaccgtttcttactgtcccatctcattcctgatttttctcccggtcctacccatttctggtggtctggtctatcctccatcaagtaagtctgcgttcagggaagaagaagcgttctggacgcaggtctcagcgcgaagaacagctaccactgaagtcacgactgtaactaaagacaagctgcgtggttactttctttccccaggagcagctccgccatttagcttgatgctcccgtgagtcacgcgtgcttttcagggcagaattaagtggtctaattctttccatttgagatttgcagttcatttgttatgggacgtctctacagctctcttttcctaaacttttgattctccggatcatgtccgtaattgtccaaggatggaattgccttaattacacttctgtcaatcatgttttcgtacctgcccagacatgagccttcatgttacaaaccttgcctcatgtggtgtgaaagcctctgtgtcttattctcactcggtccagggtttccaaatcagtgttttggggtcatgttaggggctctaggtgccccccaccctgcatatagcacatgcccatagtcaagttatctttgaccttctcagacagaatgttttggattctgagaccccctcactgtatactttgcacattgcagcctggcttgggcgccggcggtcaggtgcccatctaacgcatagaatctcgtcttctccgacatagaagggagggagaaaatttcaggttaggtcttgactctgccgccgacaggcaatgtcaccttagccatctcatttcagttttcacatctgtgctgatgtcactggatggtcataagaactgcatgaaaaagtgtgagaacatcttgaaaacttgtgaagcaccgtaaagattcactacttattttaaagaaatgttgctgatggtggggcctctgggcggctcagtcagttaagcatctgccttcagctcaggtcataatcctggagtcctagagtcaagccccaagtcatagggctccctgcccagtgggaaatctgattctccctttgcccctcccccgctccttctctctccctctttcactcattccatctctcaaatacataaagtctcaaaaaaaaaaaagttttagatgggataaatacaaggggaggatgcagaaggcagtgctcactggtcagtagttggaacagtggcccagaaagcaggactgctttgccacagagcagttctgttacctggggcacatctctaactgcttggtctcagtgttcttatccggagtgaagagcttggtccttacagctctcatattccatcctattatttttggtgttaatatcttctctttcgttttggatattcatgtctagactattcccagtggaggaaatataaagtataaattaagaccatgggggtcacacaatgatgagctgttgaagtctatgggagcagaggaacttcctgggtatagttcttttttttttttatatacatttattttttattggtgttcaacttgccaagatatagaataacacttcctgggtcgagttcacacaaataacttatagacatgatcctcccatttggcagctcagagtcaaaacaaaacagaacaaaacaaaacaacaacaacaacaacaaaactaccagtgctggcacaagcaggtaagtggataaagtatccaaatctttggaaaatggcaagttagcaggttgttgacaactgaggtgtaaaagaatagcaagctcttcaaatgcttcccaacattttagcagaacctccttgtttgtactcatgcatgtaagtgatctctgcatagttatgatgggaaactgcagtccgtgatgtctttagcagtttctagcacagactgatgtttgttattggtgctaaacaaatgtgtacttaatctgctgcccatagcagattgcctggtggcactctgagtgctaactgaggtcagtagggctcccattcttgggtaggatcaaacttcagctccagtggtttacattggctccaccggggaatccagttctagttaggctctctgctctggtgcttcagggcttcaaacaaggttttaccttttctataaaggtctttagagaaatactcaaatgtcttaatgtctatatattgagacagaaataattagtattcagatgtgtcttcagttggaaggaatacatgtatataatgacatggcaaatatctgtaagtgagccaatccctggaatcccagcacaaagtcccctccacaaaataacctgtgtaattattgtatggtattatataggaactctttctgcttatgtagggactgtatttgatgtaaaattttatgtgttcaaagaaatcattaataaaatggaaggactaggccacccttttgaactgatcattctgttgtgtgatattcagagcatttttggtcttcttgccaatttgtcagcaactgctgcagaatgagaaggtacattcattagtgatgaagtttccttgactgtagttatcatctgtatgatggccttttctcagttcaaaatgatttaactcctgaaaataaagtgaaattgtaaacttgacattaaaaaaaaaaaaaaaaaaaaaaa

SEQ ID NO: 2 - protein, 325 aa, Canis familiaris
MNSSFHLLFLDLNSNATEGNFSGPNVKNKSSPCEEMGIAVEVFLTLGLISLLENILVIGAIVKNKNLHSPMYFFVCSLAVADMLVSMSNTWETITIYLIYNKHVVIADAFVRHIDNVFDSMICISVVASMCSLLAIAVDRYVTIFYALRYHHIMTVRRSRVIITCIWTFCTGCGIVFIVYYESTYVIVCLISMFFTMLFLMVSLYIHMFLLARTHVKRIAALRGCSSVRQTASMRGAVTLTMLLGIFIVCWAPFFLHLILMISCPQNFYCSCFMSYFNMYLILIMCNSVIDPLIYAFRSQEMRKSFKEIICCHAFRIPRRFLSRY

SEQ ID NO: 3 - DNA, 978 bp, Canis familiaris
atgaattcctcatttcacttgcttttcttggatctcaactcgaatgccacagagggcaacttttcaggaccaaatgtcaagaacaagtcttcgccgtgtgaagagatgggcattgctgtggaggtgtttctgactctaggtctcatcagcctcttggagaacatcctggtcataggagccatagtgaagaacaagaacctgcactcccccatgtatttctttgtgtgcagcttagcggtagctgacatgctagtgagcatgtctaacacctgggagaccatcaccatatacttaatatataataagcacgtggtgatagcagatgcttttgtgcgtcacattgacaacgtgttcgactccatgatctgcatttccgtggtggcttccatgtgcagcttgctggccatcgcggtggacaggtacgtcaccatcttctacgccctgcgctaccaccacatcatgaccgtgaggcgttccagagtgattatcacgtgtatctggaccttttgcacaggctgcggcatcgttttcatcgtctactacgagtccacgtatgtcatcgtttgcctcatctccatgttcttcaccatgctcttcctcatggtgtccctgtacatacacatgttcctcctggcgcggactcacgtcaagcggatagcagctctgcgtggctgcagctctgtgaggcagacggccagcatgagaggggccgtcaccctgaccatgttgctgggcatcttcatcgtgtgctgggctccgttcttcctccatctcatcttgatgatttcctgcccccagaacttctactgttcttgctttatgtcttactttaatatgtacctcatactcatcatgtgtaattctgtcatcgaccctctgatatatgccttccgcagccaggagatgaggaagtccttcaaagagatcatttgttgccacgctttccgaataccccgtaggttccttagcaggtattaa

(19) United States
(12) Patent Application Publication
(10) Pub. No.: US 2003/0110518 A1
(43) Pub. Date: Jun. 12, 2003

(54) Melanocortin-5 receptor sequences and uses thereof

(76) Inventors: Karen L. Houseknecht, Old Saybrook, CT (US); Alan S. Robertson, Old Lyme, CT (US); Yongling Xiao, Pawcatuck, CT (US)

Correspondence Address: CONNOLLY BOVE LODGE & HUTZ, LLP, 1220 N MARKET STREET, P O BOX 2207, WILMINGTON, DE, 19899

(21) Appl. No.: 10/256,089

(22) Filed: Sep. 25, 2002

Related U.S. Application Data
(60) Provisional application No. 60/325,646, filed on Sep. 28, 2001.

(51) Int. Cl. A01K 67/00; C12Q 1/68; G01N 33/53; G01N 33/567; C07H 21/04; C12P 21/02; C12N 5/06; C07K 14/72

(52) U.S. Cl. 800/8; 435/6; 435/69.1; 435/320.1; 435/325; 530/350; 536/23.5; 435/7.2

(57) ABSTRACT

The present invention relates to nucleic acids encoding functional canine melanocortin 5 receptors and their polypeptide products. The present invention further relates to screening assays to identify compounds that modulate the activity or expression of the melanocortin 5 receptor of the invention. The present invention also relates to methods and therapeutic compositions for the treatment of inappropriate food intake and skin disorders comprising administering to animals compounds that modulate the activity or expression of melanocortin 5 receptors.

This application claims the benefit of U.S. Provisional Application No. 60/325,646, filed Sep. 28, 2001, which is hereby incorporated herein in its entirety.

### FIELD OF THE INVENTION

The present invention relates to nucleic acids encoding functional canine melanocortin 5 receptors and their polypeptide products. The present invention further relates to screening assays to identify compounds that modulate the activity or expression of the melanocortin 5 receptor of the invention. The present invention also relates to methods and therapeutic compositions for the treatment of inappropriate food intake, skin disorders, and metabolic disorders comprising administering to animals compounds that modulate the activity or expression of melanocortin 5 receptors. In one aspect, the invention relates to methods and compositions that agonize the activity or expression of melanocortin 5 receptors in order to treat obesity, diabetes, and dry skin conditions in companion animals, such as cats and dogs, and in agricultural animals, the methods comprising administering an agonist of the activity or expression of the melanocortin 5 receptor of the present invention. In another aspect, the invention relates to methods and compositions that antagonize the activity or expression of melanocortin 5 receptors in order to treat disorders associated with reduced caloric intake, such as anorexia and cachexia, and skin disorders, such as seborrhea and allergic dermatitis, in companion animals, such as cats and dogs, and agricultural animals, comprising administering a compound that antagonizes the activity or expression of the melanocortin 5 receptor of the present invention.

### BACKGROUND OF THE INVENTION

The control of diseases and physiological dysfunctions is of great practical importance in the care and rearing of companion and agricultural animals. Many companion animals suffer from obesity and from diseases associated with obesity, including diabetes, cardiovascular disease, cancer, and hypertension. With the recent emphasis on reduced fat consumption by people, it is increasingly recognized that agricultural animals may also be pointlessly obese. Agricultural feed that is needlessly converted to adipose tissue is an economic waste. Thus hogs that were once the primary biological source of cooking fats are now bred to be leaner. Similarly, metabolic energy expended by dairy cattle for production of butterfat can be more advantageously used for biosynthesis of milk protein and fluid. Diseases associated with obesity and aberrant lipid metabolism can also impact long-lived agricultural animals such as dairy cattle.

Despite intensive study, the regulation of animal obesity is still poorly understood. At the anatomic level, the hypothalamus regulates fat storage by controlling food intake and whole-body metabolic rate. Experimental ablation of the lateral hypothalamus reduces body fat. Bray et al., 1990 Frontiers in Neuroendocrinology 11:128-181. Conversely, damage to the ventromedial nucleus (hereinafter “VMH”) or paraventricular nucleus (hereinafter “PVN”) in the hypothalamus produces massive obesity. Id.

Recent research efforts have focused on the molecular mechanisms regulating appetite, body fat stores, energy metabolism and nutrient balance. They have revealed a complex feedback system involving many endocrine, neuroendocrine and metabolite mediators. Flier et al., 1998 Cell 92:437-440. Attempts are now being made to understand the role played by the melanocortins in energy metabolism and obesity. Melanocortins (a term which includes a variety of different peptide products resulting from post-translational processing of pro-opiomelanocortin) are known to have a broad array of physiological actions. Aside from their well-known effects on adrenal cortical function (e.g., by adrenocorticotropic hormone (hereinafter “ACTH”)), and on melanocytes (e.g., by α-melanocyte stimulating hormone (hereinafter “α-MSH”)), melanocortins have been shown to affect behavior, learning and memory, control of the cardiovascular system, analgesia, thermoregulation, and the release of other neurohumoral agents including prolactin, luteinizing hormone, and biogenic amines. Peripherally, melanocortins have been identified to have immunomodulatory and neurotrophic properties and to be involved in events surrounding parturition.

The melanocortins mediate their effects through melanocortin receptors (hereinafter “MCRs”)—a subfamily of G-protein coupled receptors. U.S. Pat. No. 5,622,860. The MCR family includes five (5) subtypes that are mostly expressed in various areas of the brain. Adan and Gispen, 1997 Peptides 18:1279-1287. The first MCRs cloned were the human and mouse melanocyte MSH receptor, MC1R, and the human adrenocortical ACTH receptor, MC2R. Mountjoy et al., 1992 Science 257:1248-1251; Chhajlani and Wikberg, 1992 FEBS Lett. 309:417-420. Subsequently, three additional melanocortin receptor polynucleotide sequences were cloned that recognize the core heptapeptide sequence (i.e., the peptide sequence MEHFRWG using standard one-letter amino acid abbreviations) of melanocortins. Two of these receptors are expressed primarily in the brain, namely MC3R (Roselli-Rehfuss et al., 1993 Proc. Natl. Acad. Sci. USA 90:8856-8860; Gantz et al., 1993 J. Biol. Chem. 268:8246-8250) and MC4R (Gantz et al., 1993, supra; Mountjoy et al., 1994 Mol. Endo. 8:1298-1308). A fifth melanocortin receptor, MC5R (originally called MC2R), is expressed in numerous peripheral organs as well as the brain. Chhajlani et al., 1993 Biochem. Biophys. Res. Commun. 195:866-873; Gantz et al., 1994, Biochem. Biophys. Res. Commun. 200:1214-1220. The native ligands and functions of these latter three receptors remain unknown.

Because of their “orphan” status as receptors without an identified native ligand, and the paucity of known physiological roles for these new receptors, investigators have attempted to characterize the receptors in vitro, by their ability to bind and respond to a variety of known melanocortins (see, e.g., Roselli-Rehfuss, 1993, supra; and Gantz, 1993, supra) or to bind to agonists and antagonists derived from MSH and ACTH amino acid sequences (see, e.g., Hruby et al., 1995, J. Med. Chem. 38:3454-3461; and Adan et al., 1994 Eur. J. Pharmacol. 269:331-337). In another approach, the members of the melanocortin receptor family were differentiated on the basis of their pattern of tissue distribution as a means for hypothesizing a function. See, e.g., Gantz et al., 1993, supra; and Mountjoy et al., 1994, supra. For example, expression of MC1R and MC2R is localized to melanocytes and to adrenal cortical cells, respectively. MC3R and MC4R are expressed primarily in the brain but not in the adrenal cortex or melanocytes. MC4R is not expressed in the placenta, a tissue that expresses large amounts of MC3R. MC5R mRNA expression is found in brain, lacrimal glands, Harderian glands, preputial gland, prostate gland, spleen, thymus, and adrenal glands. Van der Kraan et al., 1998, supra; Griffon et al., 1994 Biochem. Biophys. Res. Comm. 200:1007-1014; Fathi at al., 1995 Neurochem. Res. 20:107-113. Some, but not all investigators have observed MC5R in pancreas. Van der Kraan et al., 1998 Endocrinology 139:2348-2355; Akbulut at al., 2001 Biochem. Biophys. Res. Comm. 281:1086-1092. The protein for MC5R was identified in lachrymal gland, adipose tissue, adrenals, thymus, and spleen, but not pancreas, by Western blots using dual antibodies directed to amino-terminal and carboxy-terminal polypeptides. Id.

More particularly, the function of the MC5 receptor has been evaluated by gene ablation. Chen et al., 1997 Cell 91:789-798. Evidence from MC5R knock-out mice indicates that the MC5R has roles in the function of several exocrine glands, including sebaceous glands, lachrymal glands, Harderian glands, and the preputial gland. Id. MC5R is required for melanocortin-regulated protein secretion by the extra-orbital lachrymal glands. Id. Melanocortin peptides are known to increase total protein secretion 3-4 fold from lachrymal glands. Janh et al., 1982 European J. Biochem. 126:623-629; Leiba at al., 1990 European J. Pharmacol. 181:71-82. Moreover, mice lacking the MC5R secrete lesser amounts of lipid, including a sterol ester, from sebaceous glands. Chen, supra. The reduced secretion of lipids contributes to decreased water repulsion and impaired thermoregulation. Id. The evidence from knock-out mice is supported by immunoblot studies on rat tissues using antibodies to both amino-terminal and carboxy-terminal sequences of MC5R, which antibodies identified an apparent 77 kDa protein in lachrymal glands, adipose tissue, adrenals, thymus and splenic lymphocytes. Akbulut at al., 2001, supra.

MC5R is a 7-transmembrane spanning G-protein coupled receptor (hereinafter “GPCR”), which upon agonist activation stimulates CAMP accumulation (see Gantz at al., 1994 supra; Labbe at al., 1994 Biochemistry 33:4543-4549; Mountjoy et al., 2001 Physiol. Genomics 5:11-19). Moreover, melanocortin agonists were found to induce a transient rise in intracellular free calcium (“[Ca²⁺]_{I}”) in cells expressing MC1R, MC3R, MC4R, or MC5R. The [Ca²⁺]_{I} rise results from release from intracellular stores. Mountjoy et al. 2001, supra. To date, MC5R or the nucleotide sequences encoding MC5R have been isolated from rat (Griffon et al., 1994, supra; GenBank Accession Nos. NM013182 and L27081), human (Gantz et al., 1993, supra; GenBank Accession Nos. HUMMC5R, HSMELRECA, HSU08353, NM005913, and XM008685), chimpanzee (GenBank Accession No. AF208691), mouse (Gantz et al., 1994 supra; Labbe at al., 1994 supra; GenBank Accession Nos. L22527, NM013596, MMGMC5R, and MMU08354), swine (GenBank Accession No. AF133793), cattle (GenBank Accession No. AJ002024), and chicken (Takeuchi et al., 1998 Gen. Comp. Endocrinol. 112:220-231; GenBank Accession No. AB012868). Based upon exocrine gland dysfunction observed in MC5R-deficient mice, a role for the MC5R in the function of several exocrine glands other than pancreas was proposed (Chen et al., 1997, supra).

The melanocortin receptors respond to the same agonists, but differ in response to antagonists. For example, the effective concentration (EC₅₀) for α-MSH and desacetyl-α-MSH with regard to Ca²⁺ mobilization are not significantly different for MC1R, MC3R, MC4R and MC5R. Mountjoy et al., 2001, supra. Moreover, MC5R expressed in transformed cell lines binds α-MSH and ACTH₁₋₂₄ with similar affinity. Griffon at al., 1994, supra. An agent known as agouti protein antagonized MC4R function, but no significant antagonism of α-MSH stimulation of MC5R expressed in HEK293 cells was observed after treatment with agouti protein. Mountjoy et al., supra. Agouti-related protein (AGRP) is another antagonist of melanocortin action. AGRP and variants of AGRP block the stimulation of cAMP levels by α-MSH mediated by MC3R or MC4R, but has little effect on MC5R-mediated responses. Yang et al., 1999 Mol. Endocrinol. 13:148-155. In support of these observations, MC3R and MC4R bind a truncated form of AGRP (AGRP₈₇₋₁₃₂) and [Leu127Pro]AGRP, but the affinity for MC5R appears to be lower than for MC3R or MC4R. Yang et al., 1999, supra.

Relationship between obesity and MC5R. Obesity is observed in mutations of the pro-opiomelanocortin gene leading to low melanocortin peptide levels, in mutations of the MC3R or MC4R genes resulting in non-functional receptors, and in mutations that increase MCR antagonists (such as agouti protein or agouti-related protein). Krude et al., 1998 Nature Genetics 19:155-157; Yaswen et al., 1999 Nat. Med. 5:1066-1070; Butler et al., 2000 Endocrinology 141:3518-3521; Huszar at al., 1997 Cell 88:131-141; Yeo et al., 1998 Nature Genetics 20:111-112; Vaisse et al., Nature Genetics 20:113-114; Bultman et al., 1992 Cell 71:1195-1204; Ollmann et al., 1997 Science 278:135-137. The obesity in these aberrant conditions may result from an enhanced caloric efficiency. Ste Marie et al., 2000 Proc. Natl. Acad. Sci. USA 97:12339-12344; Butler at al., supra. Administration of a melanocortin agonist to mice lacking α-MSH results in weight loss. Yaswen at al., supra. The adipocyte-derived hormone leptin is a regulator of body weight, the absence of which leads to obesity. Intracerebroventricular administration of melanocortin receptor agonist to mice genetically deficient in leptin leads to reduced food intake and decreased body weight. Hohmann et al., 2000 Am. J. Physiol. Regulatory Integrative Comp. Physiol. 278:R50-R59. The effects are blocked by a melanocortin antagonist. Id. Moreover, peripherally administered agonist potentiates the action initiated by administration to the central nervous system, but administration of a melanocortin agonist solely to the periphery does not lead to either reduced food intake or reduced body weight. Id.

The relationship between obesity and MC5R was evaluated in a genetic linkage and association study. In the Quebec Family study linkages were observed in males between MC5R and each of body mass index, skin fold thickness, fat mass, and resting metabolic rate. Chagnon et al., 1997 Mol. Med. 3:663-673. In the same study, by comparison, an association was observed in females between MC5R and body mass index. Id.

Reproductive incompetence. Intracerebroventricular administration of melanocortins induces several behaviors, including grooming, stretching, yawning, and penile erection. Poggioli et al., 1995 Peptides 16:1263-1268; Argiolas et al., 2000 Brain Res. Bull. 51:425-431. Systemic administration of a non-selective melanocortin receptor agonist to men suffering erectile dysfunction resulted in sustained penile rigidity, yawning, and nausea, the latter often severe. Wessells, 2000 Int. J. Impot. Res. 12:S74-S79. In male rats, administration of a specific MC4R antagonist prevented the melanocortin-induced grooming, stretching, and yawning responses, but not the penile erection, suggesting that a melanocortin receptor other than MC4R is responsible. Argiolas et al., supra.

Sebaceous gland and other exocrine gland function. MC5R gene knock-out mice are deficient in several functions of exocrine glands. Chen et al., 1997, supra; U.S. Pat. No. 6,278,038. A deficiency in skin oil production, specifically sterol ester secretion from sebaceous glands, in MC5R knock-out mice leads to reduced water repellency of hair and skin and impairment of the insulating property of fur. Id. The sebotropic effect of α-MSH is thus thought to be meidated by MC5R in the cells of the sebaceous glands in the hair follicle apparatus. Abdel-Malek, 2001 Cell. Mol. Life Sci. 58:434-441. Among other changes noted in MC5R knock-out mice is altered porphyrin metabolism in Harderian glands, decreased protein secretion in tears from lachrymal glands, and decreased pheromone secretion in the Harderian and preputial glands. Chen et al., 1997, supra; U.S. Pat. No. 6,278,038.

Endotoxemia, inflammation and fever. Melanocortins have broad anti-inflammatory effects. Khoda at al., 1998 Curr. Opin. Nephrol. Hypertens. 7:413-417. Stimulation of macrophages with bacterial lipopolysaccharide (endotoxin) induces production of the inflammatory cytokine in a process that is inhibited by treatment with α-MSH. Taherzadeh et al., 1999 Am. J. Physiol. 276:R1289-R1294. Moreover, human macrophage, cellular mediators of inflammation, express MC5R. Id. When administered systemically in a model of lipopolysaccharide-induced fever, α-MSH completely prevented the onset of fever and also suppressed fever, but did not affect normal body temperature in control, afebrile rats. Huang et al., 1998 Am. J. Physiol. 275:R524-R530. II-1-induced fever is also blocked by melanocortins, but blockade of the MC3R and MC4R does not inhibit the melanocortin effect. Lawrence et al., 2001 J. Neuroendocrinol. 13:490-495. The protective effect of α-MSH on inhibiting ischemic acute renal failure may be due to its antipyretic or anti-inflammatory effects, or to direct action on the kidney. Kohda et al., 1998, supra.

Thus, evidence suggests that MC5R plays a role in regulating obesity, reproductive dysfunction, inflammation, skin coat lipid levels, and metabolic rate. There is a need for the identification of MC5R agonists and antagonists to treat disorders relating to these conditions and physiological functions. The instant invention addresses these needs by providing research tools useful for identifying agonists and antagonists and methods of treatment. In particular, the instant invention provides canine MC5R polypeptides and nucleic acids encoding the same, methods of using the same to identify agonists and antagonists of MC5R, agonists and antagonists identified by these methods, pharmaceutical compositions containing the agonists and antagonists, and methods of using the agonists, antagonists and compositions to treat obesity, diabetes, cachexia, seborrhea, psoriasis, allergic dermatitis and other disorders.

### SUMMARY OF THE INVENTION

The present invention relates to an isolated nucleic acid encoding a functional MC5R, or the complement thereof, said nucleic acid comprising a nucleic acid selected from the group consisting of:

(a) a nucleotide sequence which hybridizes under conditions of moderate stringency to the coding region of SEQ ID NO:1;

(b) a nucleotide sequence which hybridizes under conditions of moderate stringency to a polynucleotide which is complementary to the coding region of SEQ ID NO:1;

(c) a nucleotide sequence which hybridizes under conditions of moderate stringency to the coding region of the canine MC5R as deposited with the ATCC and having ATCC Accession No. PTA-3455; and

(d) a nucleotide sequence which hybridizes under conditions of moderate stringency to a polynucleotide which is complementary to the coding region of the canine MC5R as deposited with the ATCC and having ATCC Accession No. PTA-3455, with the proviso that said functional MC5R is not human, bovine, chimpanzee, murine, rat, chicken or swine.

The present invention further relates to an isolated nucleic acid encoding a functional MC5R, or the complement thereof, said nucleic acid comprising a nucleic acid selected from the group consisting of:

(a) a nucleotide sequence which hybridizes under conditions of high stringency to the coding region of SEQ ID NO:1;

(b) a nucleotide sequence which hybridizes under conditions of high stringency to a polynucleotide which is complementary to the coding region of SEQ ID NO:1;

(c) a nucleotide sequence which hybridizes under conditions of high stringency to the coding region of the canine MC5R as deposited with the ATCC and having ATCC Accession No. PTA-3455; and

(d) a nucleotide sequence which hybridizes under conditions of high stringency to a polynucleotide which is complementary to the coding region of the canine MC5R as deposited with the ATCC and having ATCC Accession No. PTA-3455.

The present invention also relates to an isolated nucleic acid comprising a nucleotide sequence that:

(a) encodes a polypeptide according to SEQ ID NO:2; or

(b) encodes a polypeptide encoded by the canine MC5R clone as deposited with the ATCC and having ATCC Accession No. PTA-3455.

A further embodiment of the present invention is an isolated nucleic acid comprising a nucleotide sequence that:

(a) encodes a polypeptide according to SEQ ID NO:2; or

(b) encodes a polypeptide encoded by the canine MC5R clone as deposited with the ATCC and having ATCC Accession No. PTA-3455, wherein said nucleic acid has a nucleotide sequence according to SEQ ID NO:1, SEQ ID NO:3 or the canine MC5R clone as deposited with the ATCC and having ATCC Accession No. PTA-3455.

A preferred embodiment of the present invention is an isolated nucleic acid comprising a nucleotide sequence having more than about 87% identity to SEQ ID NO:3 or the canine MC5R clone as deposited with the ATCC and having ATCC Accession No. PTA-3455.

A further preferred embodiment of the present invention is an isolated nucleic acid comprising a nucleotide sequence encoding a polypeptide having more than about 87% identity to SEQ ID NO:2 or the polypeptide encoded by the canine MC5R clone as deposited with the ATCC and having ATCC Accession No. PTA-3455.

Yet another preferred embodiment of the present invention is an isolated nucleic acid comprising a nucleotide sequence encoding an extracellular (“EC”) domain of a canine MC5R corresponding to amino acids 1-37, 90-119, 181-185, or 265-272 of SEQ ID NO:2 or of the polypeptide encoded by the canine MC5R clone as deposited with the ATCC and having ATCC Accession No. PTA-3455.

Yet another preferred embodiment of the present invention is an isolated nucleic acid comprising a nucleotide sequence encoding a cytoplasmic (“IC”) domain of a canine MC5R corresponding to amino acids 63-70, 139-160, 213-217, or 297-325 of SEQ ID NO:2 or of the polypeptide encoded by the canine MC54R clone as deposited with the ATCC and having ATCC Accession No. PTA-3455.

Another aspect of the present invention relates to a nucleotide vector comprising a novel nucleic acid disclosed herein.

This invention further relates to an expression vector comprising such a novel nucleic acid under the control of a nucleotide regulatory element with which the nucleic acid is not naturally associated.

Another aspect of the present invention relates to a genetically engineered host cell comprising a novel nucleic acid described herein.

Yet another aspect of the present invention is a genetically engineered host cell comprising a novel nucleic acid described herein, wherein said nucleic acid is in operative association with a nucleotide regulatory element that controls expression of said nucleic acid in the host cell.

The present invention further relates to a substantially pure polypeptide encoded by a novel nucleic acid described herein.

The present invention further relates to an isolated polypeptide encoded by a novel nucleic acid described herein.

Preferred embodiments of the present invention include a substantially pure polypeptide comprising the amino acid sequence of:

(a) SEQ ID NO:2;

(b) the canine MC5R clone as deposited with the ATCC and having the ATCC Accession NO. PTA-3455;

(c) an EC domain of a canine MC5R corresponding to amino acids 1-37, 90-119, 181-185, or 265-272 of SEQ ID NO:2 or of the polypeptide encoded by the canine MC5R clone as deposited with the ATCC and having ATCC Accession No. PTA-3455; or

(d) an IC domain of a canine MC5R corresponding to amino acids 63-70, 139-160, 213-237, or 297-325 of SEQ ID NO:2 or of the polypeptide encoded by the canine MC5R clone as deposited with the ATCC and having ATCC Accession No. PTA-3455.

Preferred embodiments of the present invention include an isolated polypeptide comprising the amino acid sequence of:

(a) SEQ ID NO:2;

(b) the canine MC5R clone as deposited with the ATCC and having the ATCC Accession NO. PTA-3455;

(c) an EC domain of a canine MC5R corresponding to amino acids 1-37, 90-119, 181-185, or 265-272 of SEQ ID NO:2 or of the polypeptide encoded by the canine MC5R clone as deposited with the ATCC and having ATCC Accession No. PTA-3455; or

(d) an IC domain of a canine MC5R corresponding to amino acids 63-70, 139-160, 213-237, or 297-325 of SEQ ID NO:2 or of the polypeptide encoded by the canine MC5R clone as deposited with the ATCC and having ATCC Accession No. PTA-3455.

Yet another aspect of the present invention relates to an antibody that immuno-specifically binds a polypeptide encoded by a novel nucleic acid described herein.

This invention further relates to a method for producing a recombinant canine MC5R polypeptide, comprising:

(a) culturing a host cell transformed with an expression vector which expresses the recombinant canine MC5R polypeptide; and

(b) recovering the recombinant canine MC5R polypeptide from the cell culture.

The invention also relates to a method for isolating a recombinant canine MC5R polypeptide, comprising:

(a) culturing a host cell transformed with an expression vector which expresses the recombinant canine MC5R polypeptide in the host cell membranes; and

(b) isolating membranes comprising canine MC5R polypeptide from the host cell.

This invention further relates to a composition comprising a substantially pure polypeptide encoded by a novel nucleic acid described herein and a carrier.

Another aspect of this invention relates to a method for detecting a polynucleotide comprising a novel nucleic acid described herein, in a sample, comprising:

(a) contacting the sample with a compound that binds to and forms a complex with the polynucleotide for a period sufficient to form the complex; and

(b) detecting the complex,

so that if a complex is detected, the polynucleotide is detected.

This invention further relates to a method for detecting such a polynucleotide in a sample, comprising:

(a) contacting the sample under stringent hybridization conditions with nucleic acid primers that anneal to a polynucleotide described herein under such conditions; and

(b) amplifying the annealed polynucleotides,

so that if a polynucleotide is amplified, the polynucleotide is detected.

In a preferred embodiment the polynucleotide is an RNA molecule that encodes a functional MC5R, and the method further comprises reverse transcribing an annealed RNA molecule into a cDNA polynucleotide.

Another aspect of the present invention relates to a method for identifying a compound that binds to the polypeptide encoded by one or more of the novel nucleic acids described herein, comprising:

(a) contacting a compound with the polypeptide for a time sufficient to form a polypeptide/compound complex; and

(b) determining whether the polypeptide/compound complex is formed,

wherein if the polypeptide/compound complex is formed, then a compound that binds to the polypeptide is identified.

This invention further relates to a method for identifying a compound that binds to a polypeptide encoded by a novel nucleic acid described herein comprising:

(a) contacting a compound with the polypeptide in a cell, for a time sufficient to form a polypeptide/compound complex, wherein the complex drives expression of a reporter gene sequence in said cell; and

(b) detecting the complex by detecting reporter gene sequence expression, so that if a polypeptide/compound complex is detected, a compound that binds to the polypeptide is identified.

This invention further relates to a method of modulating activity of a polypeptide encoded by a novel nucleic acid described herein comprising contacting a cell that expresses the polypeptide in which cell the polypeptide is heterologous with a compound that modulates activity of the polypeptide for a time sufficient to modulate said activity.

This invention further relates to a method for identifying antagonists of canine MC5R, comprising:

(a) contacting a cell line that expresses a heterologous MC5R polypeptide encoded by a nucleic acid described herein with a test compound in the presence of an MC5R agonist; and

(b) determining whether the test compound inhibits binding of the MC5R agonist to the MC5R or inhibits the functional activity of the MC5R,

in which antagonists are identified as those compounds that inhibit binding of the MC5R agonist to the MC5R or inhibit the functional activity of the MC5R.

This invention further relates to a method for identifying agonists of canine MC5R, comprising:

(a) contacting a cell line that expresses a heterologous MC5R polypeptide encoded by a nucleic acid described herein with a test compound in the presence and in the absence of an MC5R agonist;

(b) determining whether, in the presence of the MC5R agonist, the test compound inhibits the binding of the MC5R agonist to the cell line; and

(c) determining whether, in the absence of the MC5R agonist, the test compound mimics the cellular effects of the MC5R agonist on the cell line,

in which agonists are identified as those test compounds that inhibit the binding but mimic the cellular effects of the MC5R agonist on the cell line.

In a preferred embodiment the cell line is a genetically engineered cell line.

This invention further relates to a method of modulating activity of an isolated polypeptide encoded by a novel nucleic acid described herein, comprising contacting the polypeptide with a compound that modulates activity of the polypeptide for a time sufficient to modulate said activity.

The invention further relates to a non-human transgenic animal, the nucleated cells of which comprise a transgene comprising a nucleic acid described herein.

The invention further relates to a transgenic animal in which expression of genomic sequences encoding a polypeptide encoded by a novel nucleic acid described herein is prevented or repressed.

The invention further relates to a transgenic animal in which a nucleic acid is an expressed transgene contained in the genome of the animal, wherein the nucleic acid encodes a functional MC5R and comprises a nucleotide sequence selected from the group consisting of:

(a) a nucleotide sequence which hybridizes under conditions of high stringency to a polynucleotide which is complementary to SEQ ID NO:3; and

(b) a nucleotide sequence which hybridizes under conditions of high stringency to the complement of the coding region of the canine MC5R as deposited with the ATCC and having ATCC Accession No. PTA-3455, with the proviso that the functional MC5R is not human, bovine, chimpanzee, murine, rat, chicken or swine.

The invention further comprises such a transgenic animal whose nucleated cells comprise a nucleotide sequence selected from the group consisting of:

(a) a nucleotide sequence which encodes a polypeptide according to SEQ ID NO:2;

(b) a nucleotide sequence which encodes a polypeptide encoded by the canine MC5R clone as deposited with the ATCC and having ATCC Accession No. PTA-3455; and

(c) a nucleotide sequence having more than about 87% identity to SEQ ID NO:3.

This invention further relates to a method for modulating the appetite and/or metabolic rate of an animal comprising administering to the animal an effective amount of an MC5R ligand.

In a preferred embodiment, the animal has an appetite-related or metabolic disorder.

In a further preferred embodiment, the disorder causes, is caused by, or is characterized by a reduction in appetite, feeding behavior or body weight, or an increase in metabolic rate, and the ligand is an MC5R antagonist.

In yet another preferred embodiment, the disorder causes, is caused by, or is characterized by an increase in appetite, feeding behavior, or body weight, or a decrease in metabolic rate, and the ligand is an MC5R agonist.

In another embodiment, the animal is a companion animal.

In another embodiment, the companion animal is a dog or cat.

In another embodiment, the disorder is cachexia, anorexia or weaning-induced inappetence and growth lag.

In another embodiment, the disorder is a metabolic disorder.

In another embodiment, the metabolic disorder is diabetes.

In another embodiment, the animal is obese.

The invention further relates to a method for treating an exocrine gland disorder, such as lacrimal gland disorders and sebaceous gland disorders comprising administering to an animal in need thereof an effective amount of an MC5R ligand.

In a further preferred embodiment, the disorder causes, is caused by, or is characterized by abnormally low skin or coat lipid levels and the ligand is an MC5R agonist.

In a further preferred embodiment, the disorder causes, is caused by, or is characterized by excessive secretion from the exocrine glands and the ligand is an MCR5 antagonist.

In another embodiment, the animal is a companion animal.

In another embodiment, the companion animal is a dog or cat.

In another embodiment, the disorder is a skin abnormality.

In another embodiment, the disorder is seborrhea, psoriasis, pruritis, or allergic dermatitis.

In a preferred embodiment, the disorder is psoriasis.

The invention further relates to a composition comprising an agonist or antagonist of canine MC5R identified by use of one of the assays disclosed herein and a pharmaceutically acceptable carrier.

### DESCRIPTION OF THE FIGURES

FIG. 1 depicts the nucleotide sequence of the canine MC5R (SEQ ID NO:1)

FIG. 2 depicts the polypeptide sequence of the canine MC5R (SEQ ID NO:2)

FIG. 3 depicts the coding nucleotide sequence of the canine MC5R (SEQ ID NO:3)

FIG. 4 depicts the polypeptide sequence of the canine MC5R (SEQ ID NO:2) with functional domains indicated.

FIG. 5 is a graph depicting the specific binding of radiolabeled NDP-MSH ligand to HEK293a cells expressing canine MC5R.

FIG. 6 is a graph depicting the functional activity of MC5R clones.

### DETAILED DESCRIPTION OF THE INVENTION

General Overview of the Invention

The present invention relates to nucleic acids encoding a functional canine melanocortin 5 receptor (hereinafter “MC5R”) and its gene products. The present invention also relates to screening assays to identify compounds that modulate the activity or expression of the MC5R of the invention. In particular, the present invention provides in vitro assays for MC5R-binding compounds using MC5R-transfected cell lines. The MC5R-transfected cell lines may further comprise a reporter gene whose level of expression is regulated by MC5R activity. The present invention further relates to pharmaceutical compositions that modulate the activity or expression of MC5R. In particular, the pharmaceutical compositions comprise agonists or antagonists of MC5R. Antagonists may act in many different ways, such as by competitively inhibiting another MC5R agonist or antagonist, by blocking the interaction of activated MC5R with its downstream signaling pathway, by inhibiting transcription of the MC5R gene, or by inhibiting processing or translation of the MC5R mRNA. Agonists may act by activating and/or enhancing the natural biological effects of the MC5R signal transduction response or its expression.

In yet another aspect, the present invention relates to methods of treating appetite-related disorders in companion animals, livestock and poultry, comprising administering pharmaceutical formulations which modulate MC5R expression or activity. In particular, pharmaceutical compositions that enhance appetite and reproductive performance, or maintenance or recovery of body weight in sick or stressed animals, or that reduce appetite and ameliorate obesity, or treat type 2 diabetes may be administered to companion animals, livestock and poultry. In another aspect, the present invention relates to methods of treating disorders associated with exocrine gland function, such as some skin-related disorders, in companion animals, livestock, and poultry, comprising administering pharmaceutical formulations which modulate MC5R expression or activity. In particular, pharmaceutical compositions that improve skin appearance and ameliorate skin abnormalities, such as seborrhea, allergic dermatitis and psoriasis, may be administered to companion animals, livestock and poultry in need thereof.

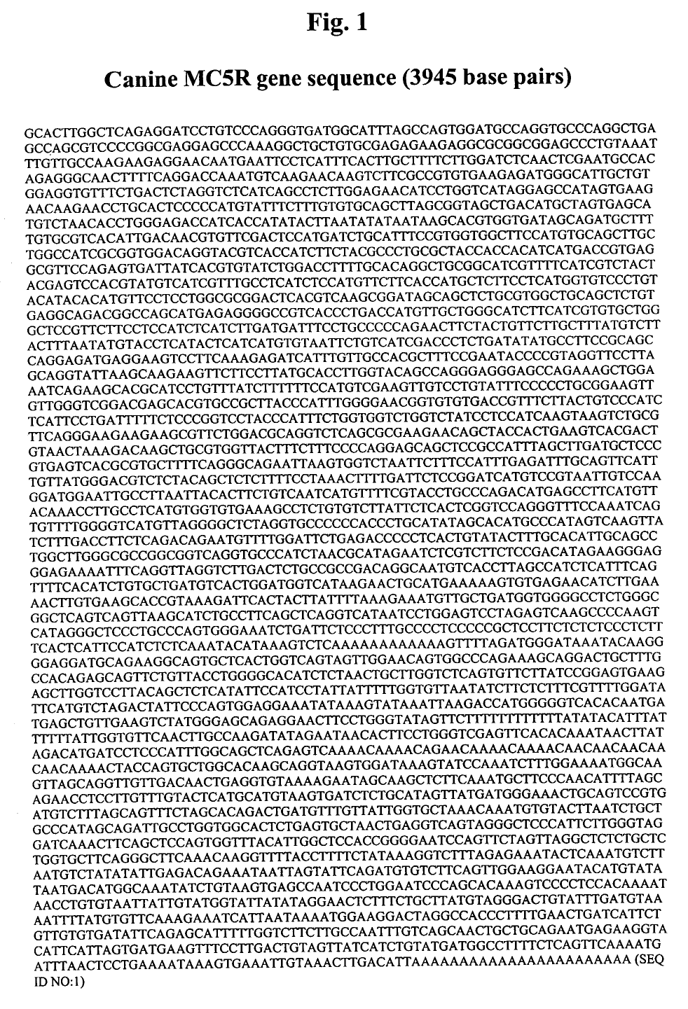

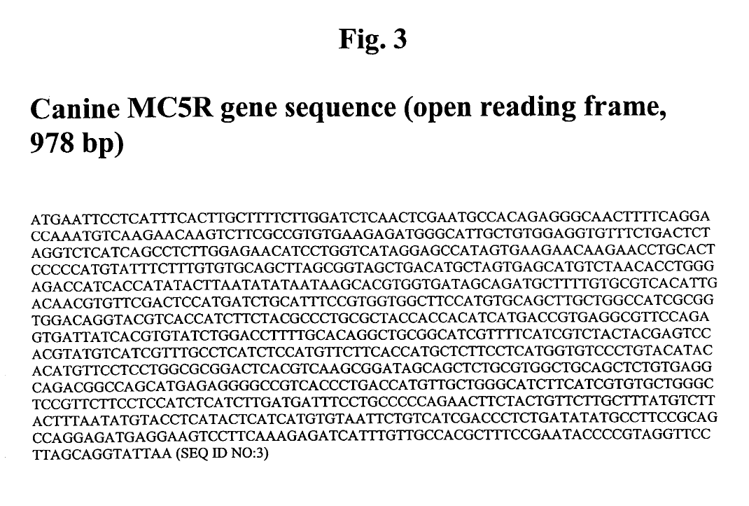

The present invention is based, in part, on the discovery of an MC5R nucleic acid that encodes the canine form of MC5R. The present invention encompasses the following: (a) isolated nucleic acids encoding canine MC5R (as shown in FIG. 1, SEQ ID NO:1 and FIG. 3, SEQ ID NO:3); (b) mutations, truncations or fragments thereof; (c) isolated polypeptides comprising canine MC5R and derivatives, peptides, and fragments thereof; and (d) recombinant fusion polypeptides comprising canine MC5R and derivatives, peptides, and fragments thereof.

Altered nucleic acids which may be used in accordance with the invention include deletions, additions or substitutions of different nucleotide residues resulting in a nucleic acid that encodes the same or a functionally equivalent gene product. The gene product itself may contain deletions, additions or substitutions of amino acid residues within the MC5R polypeptide, which result in a functionally equivalent MC5R. Such amino acid substitutions may be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. As used herein, a functionally equivalent MC5R refers to a receptor which binds to MC5R ligand or ligand analogs, such as melanocyte stimulating hormone (“MSH”), but not necessarily with the same binding affinity of its counterpart native MC5R. In addition, any nucleic acid that selectively hybridizes under highly stringent conditions or to the complement of SEQ ID NO:1 or SEQ ID NO:3, and has a gene product that is capable of binding α-MSH (Sigma, St. Louis, Mo.; BACHEM Bioscience Inc., King of Prussia, Pa.), [Nle₄,D-Phe₇]-α-MSH (hereinafter “NDP-MSH”; Sigma, BACHEM, or NEN®, Boston, Mass.), MTII (BACHEM; a melanocortin receptor agonist (Bednarek at al., 1999 Biochem. & Biophys. Res. Comm. 261:209-13)) or SHU9119 (BACHEM; a melanocortin receptor antagonist), is a subject of the instant invention, with the proviso that the nucleic acid does not encode human, chimpanzee, bovine, swine, chicken, murine or rat MC5R.

Unless otherwise indicated, all nucleic acid sequences are written 5′ to 3′, and all polypeptide and protein sequences are written N-terminal to C-terminal.

As used herein, “nucleic acid” includes DNA molecules (e.g., cDNA or genomic DNA) and RNA molecules (e.g., mRNA) and analogs of the DNA or RNA generated using nucleotide analogs. The nucleic acid can be single-stranded or double-stranded, but preferably is double-stranded DNA.

An “isolated” nucleic acid molecule is one that is substantially separated from other nucleic acid molecules which are present in the natural source of the nucleic acid. Preferably, an “isolated” nucleic acid is free of sequences that naturally flank the nucleic acid (i.e., sequences located at the 5′ and 3′ ends of the nucleic acid) in the genomic DNA of the organism from which the nucleic acid is derived. An “isolated” nucleic acid molecule, such as a cDNA molecule, can be substantially free of other cellular material, or culture medium when produced by recombinant techniques, or substantially free of chemical precursors when produced by chemical synthesis. As used herein, “polypeptide”, “peptide”, and “protein” are interchangeable. An “isolated” polypeptide is substantially free of cellular material or other contaminating proteins from the cell or tissue source in which the polypeptide is naturally found, or from which the polypeptide is derived, or substantially free from chemical precursors or other chemicals when chemically synthesized. By “substantially free of cellular material”, it is meant to include preparations of polypeptide having less than about 30% by dry weight of such cellular material or other contaminating proteins.

Isolation and Characterization of the Nucleic Acids and Polypeptides of the Invention

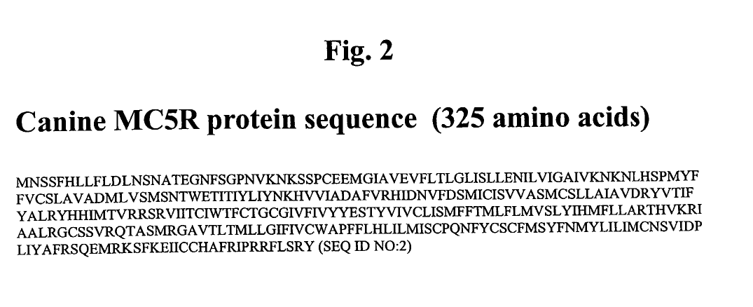

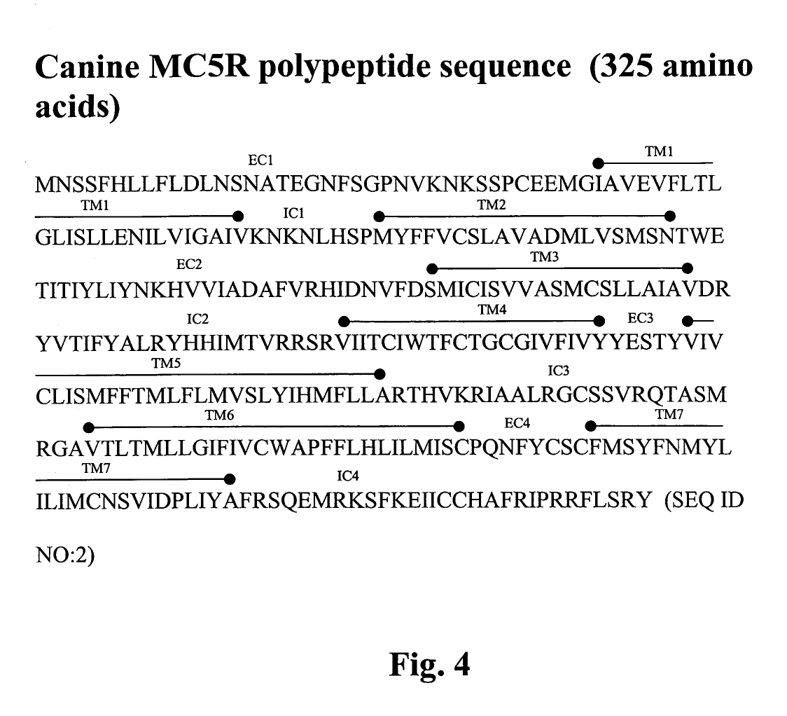

The canine derived nucleic acid sequences (SEQ ID NOS:1 and 3) encoding the deduced amino acid sequences of the canine MC5R polypeptide (SEQ ID NO:2) are shown in FIGS. 1, 2, 3, and 4. In FIG. 4, predicted transmembrane (hereinafter “TM”) domains are indicated by overbars and “TM” followed by a number. The four extracellular domains and the four cytoplasmic domains are denoted by EC1-4 and IC1-4, respectively.

The serpentine structure of the melanocortin receptors suggests that the hydrophilic domains located between the TM domains are arranged alternately outside and within the cell to form the extracellular domains (“ECs”), amino acid residues 1-37, 90-119, 181-185, and 265-272 in FIG. 4 and cytoplasmic domains (“ICs”), amino acid residues 63-70, 139-160, 213-237 and 297-325 in FIG. 4 of the receptor.

The invention relates to nucleic acids encoding canine MC5R, their gene products, variants and the gene products of these variants. Preferably, the nucleic acids of the present invention are more than about 87% identical to the canine MC5R gene sequence, as identity is calculated using the LASERGENE-MEGALIGN™ software package (DNASTAR, Inc., Madison, Wis.; percent similarity compares sequences directly, without accounting for phylogenetic relationships) which determines percent identity over the entire length of the sequences when aligned for comparison purposes. Alternatively, the preferred polypeptides of the present invention are more than about 87% identical to the canine MC5R polypeptide sequence, also using the LASERGENE-MEGALIGN™ software package. In another preferred embodiment, the polypeptides of the invention, and the gene products encoded by the nucleic acids of the invention, are functional MC5Rs that are able to bind to α-MSH, NDP-MSH, MTII or SHU9119 under physiological conditions. A more preferred embodiment is an isolated nucleic acid comprising the nucleotide sequence identified by SEQ ID NO:3 or SEQ ID NO:1.

The invention also relates to nucleic acids encoding a functional MC5R or the complement thereof that are capable of hybridizing to a nucleic acid of the invention under stringent conditions. The term “stringent” is used to refer to conditions that are commonly understood in the art as stringent. In a preferred embodiment, the nucleic acids of the invention hybridize to the canine MC5R encoding polynucleotides disclosed herein under highly stringent conditions. Procedures using such conditions of high stringency are as follows: Prehybridization of filters containing DNA is carried out for 8 h to overnight at 65° C. in buffer composed of 6× SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2× SSC, 0.01% PVP, 0.01% Ficoll, and 0.01% BSA. This is followed by a wash in 0.1× SSC at 50° C. for 45 min before autoradiography. Other conditions of high stringency which may be used are well known in the art.

In another embodiment, the nucleic acids of the invention encode a functional MC5R or the complement thereof and hybridize to a nucleic acid of the invention under moderate stringency conditions. Procedures using such conditions of moderate stringency are as follows: Filters containing DNA are pretreated for 6 h at 55° C. in a solution containing 6× SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 h at 55° C., and then washed twice for 30 minutes at 60° C. in a solution containing 1× SSC and 0.1% SDS. Filters are blotted dry and exposed for autoradiography. Washing of filters is done at 37° C. for 1 h in a solution containing 2× SSC, 0.1% SDS. Other conditions of moderate stringency which may be used are well-known in the art.

The nucleic acids of the invention may be isolated by screening of a genomic or cDNA library, such as a bacteriophage cDNA library, under conditions of reduced stringency, using a radioactively labeled fragment of a canine MC5R clone. Alternatively, a canine MC5R sequence may be used to design degenerate or fully degenerate oligonucleotide probes which may be used as PCR probes or to screen bacteriophage cDNA libraries. In another alternative, a polymerase chain reaction (PCR) based strategy may be used to clone nucleic acids encoding MC5R variants or MC5R genes from other species. Two pools of degenerate oligonucleotides, for example, corresponding to a conserved motif between the canine MC5R cDNA sequence and the MC5R sequence of another species, may be designed to serve as primers in a PCR reaction. The template for the reaction is cDNA obtained by reverse transcription of mRNA prepared from cell lines or tissue known to express MC5R. The PCR product may be subcloned and sequenced to insure that the amplified sequences represent the desired MC5R sequences. The PCR fragment may be used to isolate a full length MC5R cDNA clone by radioactively labeling the amplified fragment and screening a bacteriophage cDNA library. Alternatively, the labeled fragment may be used to screen a genomic library. For a review of cloning strategies which may be used, see e.g., Sambrook et al., 1989 Molecular Cloning, A Laboratory Manual, 2d ed., Cold Springs Harbor Press, N.Y.; and Ausubel et al., 1999 Current Protocols in Molecular Biology, (Green Publishing Associates and Wiley Interscience, N.Y.).

Alternatively, isolation of an MC5R cDNA of the invention also may be achieved by construction of a cDNA library in a mammalian expression vector such as pcDNA1 that contains SV40 origin of replication sequences which permit high copy number expression of plasmids when transferred into COS cells. The expression of MC5R on the surface of transfected COS cells may be detected in a number of ways, including the use of a labeled ligand such as MSH or a MSH agonist labeled with a radiolabel, fluorescent label or an enzyme. Cells expressing MC5R may be enriched by subjecting transfected cells to a FACS (fluorescent activated cell sorter) sort.

Of course, in addition to the exemplary methods and procedures outlined above for the isolation of the MC5R encoding polynucleotides of the invention, any other method of isolating a cDNA or genomic DNA known in the art may also be used. See, e.g., Sambrook et al., supra and Ausubel et al., supra.

Expression of the Nucleic Acids of the Invention

In accordance with the invention, an MC5R nucleic acid that encodes an MC5R polypeptide, a peptide fragment of an MC5R polypeptide, an MC5R fusion protein or functional equivalent thereof may be used to generate a recombinant nucleic acid molecule that directs the expression of an MC5R polypeptide, peptide fragment, fusion protein, or a functional equivalent thereof, in an appropriate host cell line. Alternatively, a nucleic acid that hybridizes to one or more portions of an MC5R nucleic acid also may be used in a nucleic acid hybridization assay, Southern or Northern blot analysis, etc. Of course, related nucleic acids that encode substantially the same or a functionally equivalent polypeptide may be used in the practice of the invention for the cloning and expression of the MC5R polypeptide.

A nucleic acid of the invention may be engineered in order to alter the MC5R coding sequence for a variety of ends including, but not limited to, an alteration that modifies processing and expression of the gene product. For example, a mutation may be introduced using techniques that are well known in the art, e.g. site-directed mutagenesis, to insert a new restriction site, to alter the glycosylation pattern, to alter the phosphorylation pattern, etc. For example, in certain expression systems such as yeast, a host cell line may over glycosylate the gene product. When using such an expression system it may be preferable to alter the MC5R coding sequence to eliminate all of the MC5R gene product's N-linked glycosylation sites.

In another embodiment of the invention, the MC5R nucleic acid or a modified MC5R nucleic acid may be ligated to a heterologous sequence to encode a fusion protein. For example, for screening of peptide libraries it may be advantageous to use a chimeric MC5R protein comprising a heterologous epitope that is recognized by a commercially available antibody. A fusion protein also may be engineered to contain a cleavage site located between the MC5R polypeptide and the heterologous polypeptide, so that the MC5R polypeptide can be cleaved away from the heterologous polypeptide.

In an alternate embodiment of the invention, an MC5R nucleic acid may be synthesized in whole or in part using chemical methods well-known in the art (see, e.g., Caruthers, et al., 1980 Nuc. Acids Res. Symp. Ser. 7:215-233; Crea and Horn 1980 Nuc. Acids Res. 9:2331; Matteucci and Caruthers, 1980 Tetrahedron Letters 21:719; Chow and Kempe, 1981 Nuc. Acids Res. 9:2807-2817). Alternatively, an MC5R polypeptide could be produced in whole or in part using chemical methods. For example, the MC5R polypeptide may be synthesized by solid phase techniques, cleaved from the resin, and purified by preparative high performance liquid chromatography (see, e.g., Creighton, 1983 Proteins Structures And Molecular Principles, W. H. Freeman and Co., N.Y. pp. 50-60). The composition of the synthetic polypeptide may be confirmed by amino acid analysis or sequencing (e.g., the Edman degradation procedure; see, Creighton, 1983 Proteins, Structures and Molecular Principles, W. H. Freeman and Co., N.Y., pp. 34-49).

In order to express a biologically active MC5R, an MC5R-encoding nucleic acid, or a functional equivalent thereof (as described above) is inserted into an appropriate expression vector (i.e., a vector which contains the necessary elements for the transcription and translation of the inserted coding sequence) to create an MC5R-comprising vector. An appropriate host cell line (i.e., a cell line that will allow for expression of an MC5R gene product from the MC5R-encoding nucleic acid) is transformed with the MC5R-comprising vector. The MC5R gene product and the transformed cell line may be used for a variety of purposes. These purposes include but are not limited to generating an antibody (i.e., monoclonal or polyclonal) that binds to the MC5R gene product. The antibody may, e.g., competitively inhibit binding of ligand to an MC5R receptor (see, infra), or be used for screening and selecting ligands or drugs that act via an MC5R receptor (see, infra); etc.

Methods that are well known to those skilled in the art may be used to construct expression vectors containing an MC5R nucleic acid and appropriate transcriptional/translational control signals. These methods include in vitro recombinant DNA techniques, synthetic techniques and in vivo genetic recombination. See, e.g., the techniques described in Sambrook at al., supra; Ausubel et al., supra.

A variety of host-expression vector systems may be utilized to express any one nucleic acid of the invention (the “MC5R nucleic acid”). These systems include, but are not limited to, a strain of microorganism (e.g., a strain of bacterium) transformed with a recombinant bacteriophage DNA, plasmid DNA or cosmid DNA expression vector comprising the MC5R nucleic acid; a strain of yeast (e.g., a strain of S. cerevisiae or S. pombe) transformed with a recombinant yeast expression vector comprising the MC5R nucleic acid; an insect cell line infected with a recombinant virus expression vector (e.g., a baculovirus derived expression vector) comprising the MC5R nucleic acid; a plant cell line infected with a recombinant virus expression vector (e.g., a cauliflower mosaic virus (hereinafter “CaMV”) or tobacco mosaic virus (hereinafter “TMV”) derived expression vector), or transformed with a recombinant plasmid expression vector (e.g., a Ti plasmid derived expression vector) containing the MC5R nucleic acid; an animal cell system infected with a recombinant virus expression vector (e.g., an adenovirus, adeno-associated virus or vaccinia virus derived expression vector), or an animal cell line engineered to contain multiple copies of the MC5R nucleic acid, either stably amplified (e.g., using methotrexate selection of a dhfr CHO cell line transformed with an expression vector comprising the MC5R nucleic acid and a DHFR gene) or unstably amplified in double-minute chromosomes (e.g., murine cell lines).

In a mammalian host cell line, a number of viral based expression systems may be utilized. In cases where an adenovirus is used as an expression vector, the MC5R nucleic acid may be ligated to an adenovirus transcription/translation control complex (e.g., the late promoter and tripartite leader sequence) to produce a chimeric gene. The chimeric gene then may be inserted in the adenovirus genome by in vitro or in vivo recombination. Insertion in a non-essential region of the viral genome (e.g., region E1 or E3) will result in a recombinant virus that is viable and capable of expressing the MC5R nucleic acid in an infected host (see, e.g., Logan & Shenk, 1984 Proc. Natl. Acad. Sci. USA 81:3655-3659). Alternatively, the vaccinia 7.5K promoter may be used (see, e.g., Mackett et al., 1982 Proc. Natl. Acad. Sci. USA 79:7415-7419; Mackett et al., 1984 J. Virol. 49:857-864; Panicali et al., 1982 Proc. Natl. Acad. Sci. USA 79:4927-4931).

One or more specific initiation signals also may be required for efficient translation of the MC5R nucleic acid. These signals include an ATG initiation codon and adjacent sequences. In cases where the MC5R nucleic acid comprises an entire MC5R gene, including its own initiation codon and adjacent sequences, no additional translational control signals may be needed. However, in cases where the MC5R nucleic acid does not comprise an MC5R gene's own initiation codon and adjacent sequences, one or more exogenous translational control signals, including the ATG initiation codon, must be provided. Furthermore, the initiation codon must be in phase with the reading frame of the MC5R coding sequence to ensure translation of the entire insert. Each of the exogenous translational control signals and initiation codon can be of either natural or synthetic origin. The efficiency of expression may be enhanced by the inclusion of an appropriate transcription enhancer element, transcription terminator, etc. (see, Bittner at al., 1987 Methods in Enzymol. 153:516-544).

In addition, a host cell strain may be used that modulates the expression of the MC5R nucleic acid, or modifies and processes the MC5R gene product in the specific fashion desired. Such a modification (e.g., glycosylation) or processing (e.g., cleavage) of the MC5R gene product may be important for the function of the protein. Different host cells have characteristic and specific mechanisms for the post-translational processing and modification of proteins. An appropriate cell line or host system can be chosen to ensure the correct modification and processing of the MC5R gene product. To this end, a eukaryotic host cell line that possesses the cellular machinery for proper processing of the primary transcript, and glycosylation and phosphorylation of the gene product, may be used. Examples of such a mammalian host cell line include, but are not limited to, CHO, VERO, BHK, HeLa, COS, MDCK, 293, and WI38 cell lines.

For long-term, high-yield production of a recombinant protein, stable expression may be preferred. For example, a cell line that stably expresses the MC5R nucleic acid may be engineered. Rather than using an expression vector that contains a viral origin of replication, a host cell line can be transformed with a recombinant plasmid comprising the MC5R nucleic acid controlled by one or more appropriate expression control elements (e.g., a promoter, enhancer, transcription terminator, polyadenylation site, etc.) and a selectable marker. Following transformation, the cell line may be allowed to grow for 1-2 days in an enriched medium, and then switched to a selective medium. The selectable marker in the recombinant plasmid confers resistance to the selection and allows a transformed cell to stably integrate the plasmid into a chromosome and grow to form a focus which in turn can be cloned and expanded into a cell line. This method may advantageously be used to engineer a cell line that expresses the MC5R gene product on the cell surface, and which responds to MC5R ligand mediated signal transduction. Such an engineered cell line is particularly useful in screening MC5R ligands and ligand analogs.

A host cell containing the MC5R nucleic acid and which express the biologically active MC5R gene product may be identified by any one of at least four general approaches, which are, not by way of limitation, as follows: (a) DNA-DNA or DNA-RNA hybridization; (b) the presence or absence of “marker” gene functions; (c) assessing the level of transcription as measured by the expression of MC5R mRNA transcripts in the host cell; and (d) detection of the gene product as measured by immunoassay or by its biological activity.

In the first approach, the presence of the MC5R nucleic acid inserted in the expression vector can be detected by DNA-DNA or DNA-RNA hybridization using probes comprising nucleotide sequences that are homologous to the MC5R coding sequence, respectively, or portions or derivatives thereof.

In the second approach, the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, etc.). For example, if the MC5R nucleic acid is inserted within a marker gene sequence of the vector, recombinants containing the MC5R nucleic acid can be identified by the absence of the marker gene function. Alternatively, a marker gene can be placed in tandem with the MC5R nucleic acid under the control of the same or a different promoter used to control the expression of the MC5R nucleic acid. Expression of the marker in response to induction or selection indicates expression of the MC5R nucleic acid.

In the third approach, transcriptional activity for the MC5R nucleic acid can be assessed by a hybridization assay. For example, RNA can be isolated from the transformed cell line and analyzed by Northern blot analysis using a probe identical or similar to the MC5R nucleic acid or particular portions thereof. Alternatively, total nucleic acids of the transformed cell may be extracted and assayed for hybridization to such probes.

In the fourth approach, the expression of the MC5R nucleic acid can be assessed by immunological detection of the MC5R gene product, e.g., by Western blots, immunoassays such as radioimmuno-precipitation, enzyme-linked immunoassays, etc. The ultimate test of the success of the expression system, however, involves the detection of the biologically active MC5R gene product. A number of assays can be used to detect receptor activity including, but not limited to, an MC5R ligand binding assay; and a biological assay using engineered cell lines as the test substrate.

Of course, any other approach known to one skilled in the art may be employed to identify MC5R nucleic acid comprising host cells and expression of biologically active MC5R gene product.

Screening for Compounds Modulating the Activity of the MC5R of the Invention

The aspect of the invention described in the paragraphs below encompasses screening methods (e.g., assays) for the identification of compounds that modulate the activity of the MC5R gene product. Such a compound may be an MC5R agonist or an MC5R antagonist. An MC5R antagonist causes an increase in appetite, and perhaps body weight, by reducing MC5R-dependent signaling. An MC5R agonist causes a decrease in appetite, and perhaps body weight, and an increase in metabolic rate by increasing MC5R-dependent signaling. An MC5R agonist causes an increase in lipid production from the sebaceous glands and therefore is expected to ameliorate dry coat and dry skin conditions in animals. The invention also encompasses the agonists and antagonists of MC5R identified using the disclosed assays, including small molecules (e.g., small organic compounds), large molecules (e.g., peptides), and antibodies, as well as nucleotide sequences that can be used to inhibit MC5R gene expression (e.g., antisense and ribozyme molecules), and gene or regulatory sequence replacement constructs designed to enhance MC5R gene expression (e.g., expression constructs that place the MC5R gene under the control of a strong promoter system). These compounds may be used to treat appetite, metabolic and exocrine-gland disorders in animals in need thereof.

In particular, the invention encompasses cellular and non-cellular assays that can be used to identify compounds that interact with the MC5R, e.g., modulate the activity of the MC5R and/or bind to the MC5R. The cell based assays can be used to identify compounds or compositions that affect the signal-transduction activity of MC5R, whether they bind to MC5R or act on intracellular factors involved in the MC5R signal transduction pathway. To this end, cells that endogenously express MC5R may be used to screen for compounds, preferably by engineering them to overexpress MC5R. Alternatively, cell lines, such as HEK293 cells, COS cells, CHO cells, fibroblasts, and the like, genetically engineered to express the MC5R may be used for screening purposes. The cells can be further engineered to incorporate a reporter molecule linked to the signal transduced by the activated MC5R to aid in the identification of compounds that modulate MC5R signaling activity. Preferably, a host cell line genetically engineered to express a functional receptor that responds to activation by melanocortin peptides can be used as an endpoint in the assay; e.g., as measured by a chemical, physiological, biological, or phenotypic change, induction of a host cell gene or a reporter gene, change in cAMP levels, adenylyl cyclase activity, host cell G protein activity, extracellular acidification rate, host cell kinase activity, calcium ion signaling, proliferation, differentiation, etc.

By way of example but not limitation, a cell line suitable for a cell based assay may be made, e.g., by transfecting HEK293 cells with a canine MC5R nucleic acid using any method known in the art. For example, a 50% confluent plate containing HEK293 cells can be transiently transfected with a vector construct comprising a canine MC5R nucleic acid using FuGENE 6™ (F. Hoffmann-La Roche Ltd, Basel, Switzerland) as a transfection carrier (4 μl FuGENE 6™ per ug plasmid DNA). The transfected cells may be harvested 48 hours post transfection using Sigma dissociation buffer (Sigma, St. Louis, Mo.) centrifuged and resuspended in binding buffer (50 mM HEPES, 5 mM MgCl, 0.1% BSA, and a protease inhibitor cocktail (Sigma P-8340™ (Sigma, St. Louis, Mo.), pH=7.5). The resuspended cell population is counted using a hemacytometer. A cell volume titration is performed by, e.g., pipetting varying volumes in triplicate into tubes. Either buffer or excess non-radioactive NDP-MSH (2 μM final conc.) is added. Excess non-radioactive NDP-MSH is used to ascertain non-specific binding. Radiolabled NDP-MSH ([¹²⁵I][Nle₄,D-Phe7]-α-MSH; NEN®, Boston, Mass.) is added to a final concentration of 75 pM. The reaction is incubated at 37° C. for one hour, then the cells are centrifuged at 5000×G for 10 minutes. The supernatant is aspirated, to remove unbound label, and the amount of membrane-associated (i.e., receptor-bound) label is determined using a gamma counter. One or more transfected cell lines exhibiting a high level of specific binding of labeled NDP-MSH are selected for further use.

In utilizing such cell systems, the cells expressing the melanocortin receptor are contacted with a test compound or vehicle controls (e.g., placebos). The cells then may be assayed to measure the expression and/or activity of components of the signal transduction pathway of the melanocortin receptor, or the activity of the signal transduction pathway itself may be assayed. For example, after exposure, cell lysates may be assayed for induction of cAMP. The ability of a test compound to increase levels of cAMP, above those levels seen with cells treated with a vehicle control, indicates that the test compound induces signal transduction mediated by the melanocortin receptor expressed by the host cell (i.e., the test compound is an agonist). When screening a compound that may act as an antagonist of MC5R, it is necessary to include a ligand that activates the MC5R, e.g., α-MSH, β-MSH or ACTH, to test for inhibition of signal transduction by the test compound as compared to a vehicle control.

In one specific embodiment, a cell based assay is used to determine whether a candidate ligand binds to MC5R. In an example of such an assay, cells from an MC5R transfected cell line (created, e.g., as above) are contacted with radiolabeled NDP-MSH. Other cells from the same MC5R transfected cell line are contacted with radiolabeled NDP-MSH and a candidate ligand. Each group of cells is incubated at 37° C. for one hour, then each group of cells is centrifuged at 5000×G for 10 minutes. The supernatant is aspirated, to remove unbound label, and the amount of membrane-associated (i.e., receptor-bound) label is determined using a gamma counter. A candidate ligand that displaces radiolabeled NDP-MSH from MC5R, as evidenced by a candidate ligand-dependent reduction in membrane-bound radioactivity, is selected for further analysis.

In another specific embodiment, a cell based assay is used to determine whether a candidate ligand is an agonist or antagonist of MC5R. In one such assay, an MC5R transfected cell line (created, e.g., as above) is further transfected with a reporter construct. The reporter construct is a gene comprising two characteristics. First, its level of expression is regulated by the level of activity of MC5R. For example, the reporter gene's expression can be dependent upon a cAMP-responsive element. The responsive element can increase transcription of the reporter gene in response to MC5R-dependent signaling, in which case increased expression of the reporter gene correlates with increased MC5R-dependent signaling, or the responsive element can decrease transcription of the reporter gene in response to MC5R-dependent signaling, in which case decreased expression of the reporter gene correlates with decreased MC5R-dependent signaling. Second, the reporter construct comprises a reporter gene. The reporter gene comprises a nucleic acid that encodes a gene product that can be assayed. A wide range of reporter genes may be employed. For example, the reporter gene may be a nucleic acid encoding chloramphenicol acetyltransferase (hereinafter “CAT”), luciferase, GUS, growth hormone, or placental alkaline phosphatase (hereinafter “SEAP”). Following exposure of the cells to the test compound, the level of reporter gene expression may be quantitated to determine the test compound's ability to regulate receptor activity. Particularly useful in the practice of the invention is the use of an alkaline phosphatase encoding reporter gene as this enzyme is secreted from the cell, thus tissue culture supernatant may be assayed for secreted alkaline phosphatase. In addition, alkaline phosphatase activity may be measured by colorimetric, bioluminescent or chemilumenscent assays such as those described in Bronstein at al., 1994, Biotechniques 17: 172-77. Such assays provide a simple, sensitive and easily automatable detection system for pharmaceutical screening. Other reporter genes that may be used include those that result in bioluminescence, colorimetric reactions or fluorescence. For example, the reporter gene may encode for a pigment (see, e.g., Bonhoeffer, 1995 Arzneimittelforschung 45:351-356) such as bacterial rhodopsin (see, Ng at al., 1995 Biochemistry 34:879-890), melanin (see, Vitkin et al., 1994 Photochemistry and Photobiology 59:455-62), aquorins (Molecular Probes, Eugene, Oreg.), green fluorescent protein (hereinafter “GFP”; Clonetech, Palo Alto, Calif.; Chalfie et al., 1994 Science 263:802-805; Cubitt et al., 1995 TIBS 20:448-455), yellow fluorescent protein (see, Daubner at al., 1987 Proc. Natl. Acad. Sci. U.S.A. 84:8912-8916), flavins, bioflavinoids, hemoglobin (see, Chance et al., 1995 Analytical Biochemistry 227:351-362; Shen at al., 1993 Proc. Natl. Acad. Sci. U.S.A. 90:8108-8112), heme (Pieulle et al., 1996 Biochem. Biophys. Acta 1273: 51-61), indigo dye (Murdock et al., 1993 Biotechnology 11:381-386), peridinin-chlorophyll-a protein (hereinafter “PCP”) (Ogata et al., 1994 FEBS Letters 356:367-371), or pyocyanine (al-Shibib and Kandela, 1993 Acta Microbiologica Polonica 42:275-280). Alternatively, the reporter gene may encode an enzyme that can cleave a color absorbing substrate such as β-lactamase, a luminescent or fluorescent protein, an enzyme with a fluorescent substrate, or any other gene that encodes an optically active chemical or that can convert a substrate to an optically active compound. In a further alternative, the reporter gene may encode photoproteins. In each case, the reporter gene is operatively linked to an inducible promoter which is activated by MC5R-dependent signal transduction (e.g., a cAMP responsive promoter element).

In a specific embodiment of the invention, a bioluminescent reporter gene is employed. Several types of bioluminescent reporter genes are known, including the luciferase family (see, e.g., Wood et al., 1989 Science 244:700-702). Members of the luciferase family have been identified in a variety of prokaryotic and eukaryotic organisms. Luciferase and other enzymes involved in the prokaryotic luminescent (lux) systems, as well as the corresponding lux genes, have been isolated from marine bacteria in the Vibrio and Photobacterium genera and from terrestrial bacteria in the Xenorhabdus genus, also called photorhalodus. An exemplary eukaryotic organism containing a luciferase system (luc) is the North American firefly Photinus pyralis. Firefly luciferase has been extensively studied, and is widely used in ATP assays. cDNAs encoding luciferases from Pyrophorus plagiophthalamus, another species, click beetle, have been cloned and expressed (see, Wood et al., supra). This beetle is unusual in that different members of the species emit bioluminescence of different colors. Four classes of clones, having 95-99% similarity with each other, were isolated. They emit light at 546 nm (green), 560 nm (yellow-green), 578 nm (yellow) and 593 nm (orange).

Luciferases requires a source of energy, such as ATP, NAD(P)H, and the like, and a substrate, such as luciferin, decanal (bacterial enzymes) or coelentrizine and oxygen. The substrate luciferin must be supplied to the luciferase enzyme in order for it to luminesce. Thus, a convenient method for providing luciferin is to express not only the luciferase but also the biosynthetic enzymes for the synthesis of the substrate decanal. Oxygen is then the only extrinsic requirement for bioluminescence, in bacteria expressing these proteins from the Lux operon.

For example, the lux operon obtained from the soil bacterium Xenorhabdus luminescence (Frackman et al., 1990 J. Bact. 172:5767-5773) may be used as the reporter gene, as it confers on transformed E. coli the ability to emit photons through the expression of the two subunits of the heterodimeric luciferase and three accessory proteins (Frackman et al., 1990, supra). Optimal bioluminescence for E. coli expressing the lux genes of X. luminescence is observed at 37° C. (Szittner and Meighen 1990 J. Biol. Chem. 265:16581-16587; Xi et al., 1991 J. Bact. 173:1399-1405), which contrasts the low temperature optima of luciferases from eukaryotic and other prokaryotic luminescent organisms (see, Campbell, 1988 Chemiluminescence. Principles and Applications in Biology and Medicine (Chichester, England: Ellis Horwood Ltd. and VCH Verlagsgesellschaft mbH)). Thus, the reporter gene may be chosen according to the nature and the requirements of a specific application. For example, the luciferase from X. luminescence is well-suited for use as a marker for studies in animals.

Luciferase vector constructs can be adapted for use in transforming a variety of host cells, including most bacteria, and many eukaryotic cells. In addition, certain viruses, such as herpes virus and vaccinia virus, can be genetically-engineered to express luciferase. For example, Kovacs and Mettenlieter, 1991 J. Gen. Virol. 72:2999-3008, teach the stable expression of the gene encoding firefly luciferase in a herpes virus. Brasier and Ron, 1992 Meth. in Enzymol. 216:386-96, teach the use of luciferase gene constructs in mammalian cells. Luciferase expression from mammalian cells in culture has been studied using CCD imaging both macroscopically (see, Israel and Honigman, 1991 Gene 104:139-145) and microscopically (see, Hooper et al., 1990 J. Biolum. and Chemilum. 5:123-130).

To be useful in this screening assay, the host cell line expressing functional MC5R should give a significant response to MC5R ligand, preferably greater than 5-fold induction over background. The host cell line should preferably possess a number of characteristics to maximize the response induced by melanocortin peptides: (a) a low natural level of cAMP, (b) G proteins capable of interacting with the MC5R, (c) a high level of adenylyl cyclase, (d) a high level of protein kinase A, (e) a low level of phosphodiesterases, and (f) a high level of cAMP response element binding protein would be advantageous. To increase response to melanocortin peptide, host cells could be engineered to express a greater number of favorable factors or a lesser number of unfavorable factors. In addition, alternative pathways for induction of the CRE reporter could be eliminated to reduce basal levels.

In a specific embodiment, a transfected cell line comprising both an MC5R nucleic acid and a reporter gene is contacted with the candidate ligand at 37° C. for one hour. Expression of the reporter gene is then measured and compared to reporter gene expression in a similar batch of cells treated identically but for contact with the candidate ligand. A candidate ligand causing a five-fold or greater increase in reporter gene expression is selected for further study as a potential MC5R agonist. Another version of this screen can be used to identify a potential antagonist of MC5R. In this version of the screen, prior to contacting the transfected cells with the candidate antagonist, the cells are contacted with a known agonist (e.g., NDP-MSH). A candidate ligand causing a five-fold or greater decrease in reporter gene expression is selected for further study as a potential MC5R antagonist.

When it is desired to discriminate between the melanocortin receptors and to identify compounds that selectively agonize or antagonize the MC5R, the assays described above should be conducted using a panel of host cells, each genetically engineered to express one of the melanocortin receptors (MC1R through MC5R). To this end, host cells can be genetically engineered to express any of the amino acid sequences known for melanocortin receptors 1 through 5. The cloning and characterization of each receptor from one or more organisms has been described, e.g.: murine and human MC1R and MC2R (Mountjoy, 1992, Science 257:1248-1251; Chhajlani and Wikberg, 1992, FEBS Lett. 309: 417-420); rat MC3R (see, Roselli-Rehfuss et al., 1993, Proc. Natl. Acad. Sci. USA 90: 8856-8860; Gantz et al., 1993, J. Biol. Chem. 268: 8246-8250); and murine and human MC5R (Chhajlani et al., 1993, Biochem. Biophys. Res. Commun. 195:866-873; Gantz et al., 1994, Biochem. Biophys. Res. Commun. 200:1214-1220), each of which is incorporated by reference herein in its entirety. Thus, each of the foregoing sequences can be utilized to engineer a cell or cell line that expresses one of the melanocortin receptors for use in screening assays described herein. To identify compounds that specifically or selectively regulate MC5R activity, the activation, or inhibition of MC5R activation is compared to the effect of the test compound on the other melanocortin receptors.

Alternatively, if the host cells express more than one melanocortin peptide receptor, the background signal produced by these receptors in response to melanocortin peptides must be “subtracted” from the signal (Gantz et al., 1993, supra). The background response produced by these non-MC5R melanocortin receptors can be determined by a number of methods, including elimination of MC5R activity by antisense, antibody or antagonist. In this regard, it should be noted that wild type CHO cells demonstrate a small endogenous response to melanocortin peptides which must be subtracted from background. Alternatively, activity contributed from other melanocortin receptors could be eliminated by activating host cells with a MC5R-specific ligand, or including specific inhibitors of the other melanocortin receptors.

In another aspect, the invention comprises a plurality of in vitro assays using preparations of MC5R for determining whether a test compound is an agonist or antagonist of MC5R. Such preparations of MC5R may be obtained by methods readily known in the art, see, e.g., Ausubel et al., 1988, supra. In a preferred embodiment, a group of test compounds is used serially in the in vitro assays, which together comprise a high throughput screen for MC5R agonists and antagonists. In the high throughput screen, a compound identified as a candidate agonist or antagonist by one assay in the series is used in the next assay in the series. A compound that is not identified as a candidate agonist or antagonist by one assay in the series is not used in the next assay in the series. In an especially preferred embodiment, this series of in vitro assays comprises an in vitro binding assay, a fluorescence imaging plate reader (hereinafter “FLIPR®” (Molecular Devices, Sunnyvale, Calif.)) assay, a cAMP functional assay, and an assay to determine whether the candidate agonist or antagonist binds preferentially to any of MC1R through MC5R. Each of these assays is described in turn below. Of course, the assays may be performed in different orders as described.

First, an in vitro binding assay is performed, in which compounds are screened for specific binding to canine MC5R-containing HEK293 cell membranes in vitro. Such an in vitro assay allows quantification of binding of a ligand to MC5R, which cannot be achieved using whole-cell assays (due to MC5R internalization and recycling). For this assay, canine MC5R-containing membranes are prepared from cells transfected as above using any method known in the art. In a specific, but exemplary embodiment, cells are harvested in a reaction tube using Sigma dissociation buffer, centrifuged and resuspended in ice cold homogenization buffer (1 mM EDTA, 1 mM EGTA, 10 mM HEPES and a protease inhibitor cocktail (Sigma P-8340™), pH=7.5). The resuspended cells are incubated on ice for at least 10 minutes, then homogenized with 20 strokes of a tight fitting glass/glass dounce homogenizer. The cell extracts are centrifuged at 1000×G for 10 minutes at 4° C. to pellet nuclei and unlysed cells. The supernatant fraction is transferred to a new tube and centrifuged at 25000×G for 20 minutes at 4° C., in order to pellet the plasma membrane. The supernatant fraction from this centrifugation is discarded, and the pellet is washed by resuspending it in homogenization buffer, homogenizing it with 20 strokes of a tight fitting glass/glass dounce homogenizer, and centrifuging the homogenate at 25000×G for 20 minutes at 4° C. The supernatant is again discarded, and the pellet is resuspended in a volume of homogenization buffer sufficient to yield a protein concentration of 1-5 mg/ml. 500 μl aliquots are frozen at −70° C. for long term storage. The protein concentration of the extracts may be determined using any method known in the art. For example, an aliquot can be diluted 5-10 fold and the BCA kit (Pierce, Rockford, Ill.) may be used.

The membrane preparation is then subjected to the actual MC5R-binding assay to identify test compounds that bind to canine MC5R. In this binding assay, membranes are incubated with labeled ligand in the presence or absence of test compound. Compounds that bind to the receptor and compete with labeled ligand for binding to the membranes reduce the signal compared to the vehicle control samples.

In a preferred embodiment, radiolabeled NDP-MSH, thawed canine MC5R/HEK293 membrane, and unlabeled test compound are resuspended in assay buffer (50 mM HEPES, 5 mM MgCl, 0.1% BSA, and a protease inhibitor cocktail (Sigma P-8340™), pH=7.5) are mixed to a final concentration of NDP-MSH of 0.05 nM and incubated at 37° C. for 1 hour. This binding reaction is terminated by harvesting membranes onto a UNIFILTER® GF/C® 96-well microplate (Packard Instrument Co., Meriden, Conn.) treated with polyethylimmine (Sigma-Aldrich Co., St. Louis, Mo.). Scintillant is added and a beta counting instrument is utilized. Assays can be performed in a 96 well plate format. Unlabeled test compounds are examined over a range of concentrations from 0.01 nM to 20000 nM, and IC₅₀ values are determined. A compound that “hits” in the initial binding screen (i.e., that binds to MC5R with a K_{d}<10 μM) preferably is re-tested in a 7-point dose-titration assay for determination of IC₅₀ and/or Kₗ values. The candidate compounds that show potent binding in this binding assay are then screened for functional activity in the following assays.

In alternative in vitro binding assay for MC5R agonists and antagonists, soluble MC5R may be recombinantly expressed and utilized in non-cell based assays to identify compounds that bind to MC5R. The recombinantly expressed MC5R polypeptides or fusion proteins containing one or more of the ECs of MC5R prepared as described below can be used in the non-cell based screening assays. Alternatively, peptides corresponding to one or more of the ICs of MC5R, or fusion proteins containing one or more of the ICs of MC5R can be used in non-cell based assay systems to identify compounds that bind to the cytoplasmic portion of the MC5R; such compounds may be useful to modulate the signal transduction pathway of the MC5R. In non-cell based assays, the recombinantly expressed MC5R is attached to a solid substrate such as a test tube, microtitre well or a column, by means well known to those in the art. See, Ausubel et al., supra. The test compounds are then assayed for their ability to bind to the MC5R.

As a second assay, a FLIPR® assay is performed with the candidate compounds identified to potently bind to MC5R in the above binding assay. Because MC5R proteins are members of the G protein coupled receptor (hereinafter “GPCR”) family, this assay is designed to involve a functional G-protein coupled screen. In a preferred embodiment, an MC5R is linked to the phospholipase C (hereinafter “PLC”) pathway by the use of a special G protein. The G protein may be a naturally occurring “promiscuous” G protein (i.e., a G protein which can link a plurality of GPCR types to the PLC pathway (Offermans et al., 1995, J. Biol. Chem. 270:15175-15180), or it may be a modified, chimeric G protein designed to link a selected GPCR to the PLC pathway (Conklin et al., 1996, Molecular Pharmacology 50:885-890). The activation or inhibition of a GPCR by an agonist or antagonist can be measured using either of these approaches by measuring changes in the intracellular calcium level caused by activation or inhibition of PLC.

In a specific but exemplary embodiment, a HEK293a stable cell line expressing canine MC5R (“cMC5R”) is grown in culture medium (DMEM, 10% FBS, 100 units Penicillin/Streptomycin, 300 mg/ml GENETICIN® (GIBCO BRL, Rockville Md.)), transfected with vectors carrying two different G-protein genes, Gα15 and Gα16 (see FIGS. 12 and 13), and selected for stable incorporation of these DNA using a cell medium solution containing 300 μg/ml of ZEOCIN™ (Invitrogen, Carlsbad, Calif.), using techniques well-known in the art. Alternatively, a vector containing a chimeric G-protein (e.g. Gαqi5, a Gα with the last 5 amino acids replaced with those from Gαs) can be generated and used. Single clonal colonies are selected and expanded using cloning cylinders (Sigma-Aldrich Co., St. Louis, Mo.). Individual clones are tested for the receptor's ability to couple with the incorporated promiscuous G-proteins using a FLIPR® assay, as described below. Gα15 and Gα16 are known as promiscuous G-proteins because of their ability to functionally couple to any G-Protein receptor and transduce signaling to increase intracellular calcium. A FLIPR® machine (Molecular Devices, Sunnyvale, Calif.) allows one to quantify such a calcium signaling cascade using fluorescent dyes available commercially.

HEK293a/cMC5R/Gα15, Gα16 are grown to confluence and harvested with Trypsin-EDTA (GIBCO-BRL, Rockville, Md.). Cells are resuspended in fresh culture medium containing 300 μg/ml ZEOCIN™. The cell suspension is counted using a hemacytometer and approximately 50,000 cells per well are added to poly-d-lysine-coated black/clear 96 well plates (Becton Dickinson Labware, Bedford, Mass.). Approximately 48 hours after plating, the growth medium is aspirated off, and replaced with serum-free medium containing 25 μg per 96 well plate of calcium-sensitive fluorescent dye Fluo-4 (Molecular Probes, Eugene, Oreg.) and 2.5 mM Probenicid (Sigma-Aldrich, St. Louis, Mo.). The plates are incubated for 1 hour at 37° C., after which the cells are washed 3 times with Hepes Saline solution containing 2.5 mM Probenicid to remove excess dye. The plates are then added to the FLIPR® individually, and fluorescence level is continuously monitored over a 2-minute period. A candidate agonist (in the presence or absence of antagonist), or candidate antagonist (in the presence or absence of agonist) is added to each of the 96 wells simultaneously after 20 seconds of baseline recording. A six-point dose-titration assay is performed for each compound. An increase in fluorescence is indicative of increases in intracellular calcium levels. Time-courses are exported as ASCII files to EXCEL™ (Microsoft, Redman, Wash.) and subsequently analyzed. An agonist candidate that increases intracellular calcium levels, or an antagonist candidate that decreases intracellular calcium levels, is selected for further study.

As a third assay, a cAMP functional assay is performed to test the candidate agonists or antagonists identified by the above FLIPR® assay. Any cAMP functional assay known in the art may be used. This assay is based on the MC5R's characteristic to be a receptor that, when activated by its agonist ligand, stimulates cAMP accumulation. Direct measurement of cAMP produced by a stable canine MC5R-expressing HEK293 cell line contacted with the candidate compound can determine whether the compound is an MC5R agonist or antagonist.

In a specific but exemplary embodiment the cAMP assay is performed using the Adenylyl Cyclase Activation FLASHPLATE™ Assay (NEN® Life Science Products, Inc., Boston, Mass.). An MC5R-transfected cell line is harvested using Sigma Dissociation Buffer, washed, centrifuged and resuspended in Stimulation Buffer (provided in kit). The resuspended cell population is counted using a hemacytometer. Cells are pelleted again by centrifugation and resuspended with Stimulation Buffer to 0.5-5×10⁶ cells/ml. A cell volume titration is performed by varying volumes in triplicate into wells. For each cell concentration tested, a full candidate agonist or antagonist dose titration is performed. A known agonist, such as NDP-MSH or α-MSH, may be used as a positive control. The time of agonist stimulation is also varied from 15 to 45 minutes. To stop agonist stimulation, detection mix is added at the appropriate time to all wells. Each plate is covered and incubated 20 hours at room temperature, then placed in a Wallac 1450 MICROBETA™ multidetector (PerkinElmer Life Sciences, Gaithersburg, Md.) for scintillation counting. Other cAMP assay kits can be used such as the cAMP SPA™ kit (RPA559; Amersham Pharmacia Biotech, Inc., Piscataway, N.J.) or other kits by other vendors.

In a fourth assay, the candidate MC5R agonists or antagonists may be tested to determine whether it binds preferentially to MC5R or another melanocortin receptor (“MCR”) using any method known in the art. In a preferred embodiment, a candidate agonist or antagonist that exhibits potency in the in vitro binding and functional screens described above is assayed for selectivity of binding to MC5R versus another MCR receptor. One cell line that expresses MC5R and another cell line that expresses another MCR are created using an HEK293 cell line as described above. For each of these transfected cell lines, whole cell and membrane fraction binding assays are performed as described above. For a more thorough investigation of receptor number, ligand affinity and for comparison to current procedures and literature values, a saturation binding study is performed using membrane fractions. The protein concentration of each preparation is determined so that it can be normalized to the protein concentration of a specific membrane preparation using the BCA protein assay kit. A constant amount of membrane fraction (as measured by total protein amount) is used for each membrane fraction preparation (for a membrane fraction from an MC4R-expressing cell line, this amount is 30 μg protein; for a membrane fraction from an MC3R-expressing cell line, this amount is 70 μg). A saturation binding isotherm is determined by titrating in various amounts of a radioactively labeled candidate agonist or antagonist. The dissociation constants (hereinafter “K_{d}”) and the maximum number of specific binding sites per mg of membrane protein (hereinafter “Bₘₐₓ”) for binding of the candidate agonist or antagonist to MC5R and the other MCR are determined by saturation binding isotherm and non-linear regression analysis using the software package GraphPad PRISM® (GraphPad Software Inc., San Diego, Calif.). A linear Scatchard line verifies that each membrane preparation contains a population of receptors having a single affinity for the radioactive ligand.

In vitro cell based assays also may be designed to screen for compounds that modulate MC5R expression at either the transcriptional or translational level. In one embodiment, a nucleic acid encoding a reporter gene (see, supra) may be linked to a regulatory element of the MC5R gene and used in appropriate intact cells, cell extracts or lysates to identify compounds that modulate MC5R gene expression. Appropriate cells or cell extracts are prepared from any cell type that normally expresses the MC5R gene, thereby ensuring that the cell extracts contain the transcription factors required for in vitro or in vivo transcription. The screen may be used to identify compounds that modulate the expression of the reporter construct. In such screens, the level of reporter gene expression is determined in the presence of the test compound and compared to the level of expression in the absence of the test compound.

To identify compounds that modulate MC5R translation, cells or in vitro cell lysates containing MC5R transcripts may be tested for modulation of MC5R mRNA translation. To assay for inhibitors of MC5R translation, test compounds are assayed for their ability to modulate the translation of MC5R mRNA in in vitro translation extracts.

Compounds that decrease the level of MC5R expression, either at the transcriptional or translational level, may be useful for treatment of decreased appetite-related disorders such as anorexia and cachexia or for treatment of disorders involving excessive secretion of sebum, such as seborrhea. In contrast, those compounds that increase the expression of MC5R may be useful for treatment of increased appetite-related disorders such as obesity, and for treatment of disorders involving inadequate lipid levels in the skin or coat.

The assays described above can identify compounds that affect MC5R activity. For example, compounds that affect MC5R activity include but are not limited to compounds that bind to the MC5R, inhibit binding of natural ligands, and either activate signal transduction (agonists) or block activation (antagonists), and compounds that bind to a natural ligand of the MC5R and neutralize ligand activity. Compounds that affect MC5R gene activity (by affecting MC5R gene expression, including molecules, e.g., proteins or small organic molecules, that affect transcription or interfere with splicing events so that expression of the full length or the truncated form of the MC5R can be modulated) also can be identified using the screens of the invention. However, it should be noted that the assays described also can identify compounds that modulate MC5R signal transduction (e.g., compounds which affect downstream signaling events, such as inhibitors or enhancers of G protein activities that participate in transducing the signal activated by ligand binding to the MC5R). The identification and use of such compounds that affect signaling events downstream of MC5R and thus modulate effects of MC5R on the development of body weight disorders and skin disorders are within the scope of the invention. In some instances, G protein-coupled receptor response has been observed to subside, or become desensitized with prolonged exposure to ligand. In an embodiment of the invention, assays may be utilized to identify compounds that block the desensitization of MC5R. Such compounds may be used to sustain the activity of MC5R, and can be used as part of a therapeutic method for the treatment of appetite disorders and skin disorders.

Compounds identified via assays such as those described herein may be useful for ameliorating appetite disorders, metabolic disorders, and skin disorders in animals. The efficacy of such compounds can be tested in an animal model system. For example, an animal model may be exposed to a compound suspected of exhibiting an ability to ameliorate appetite disorder symptoms, at a sufficient concentration and for a time sufficient to elicit such an amelioration of appetite disorder symptoms in the exposed animal. The response of the animal to the exposure may be monitored by assessing the reversal of symptoms associated with appetite disorders such as cachexia or obesity. Dosages of test agents may be determined by deriving dose-response curves, as discussed below.

The animal model may be an animal that overexpresses the MC5R gene product. Animals of any species, including, but not limited to, mice, rats, rabbits, guinea pigs, pigs, micro-pigs, goats, cattle, and non-human primates, e.g., baboons, monkeys, and chimpanzees may be used to generate MC5R transgenic animals. Transgenic mice and rats are especially preferred because of their relatively small size, ease of care, short generation time, extensively studied genetic constitution, and well-known laboratory rearing conditions.

Any technique known in the art may be used to introduce the canine MC5R transgene into an animal to produce the founder line of a transgenic animal. Such techniques include, but are not limited to pronuclear microinjection (see, Hoppe and Wagner, 1989, U.S. Pat. No. 4,873,191); retrovirus mediated gene transfer into germ lines (see, Van der Putten et al., 1985 Proc. Natl. Acad. Sci. USA 82:6148-6152); gene targeting in embryonic stem cells (Thompson et al., 1989 Cell 56:313-321); electroporation of embryos (Lo, 1983 Mol Cell. Biol. 3:1803-1814); and sperm-mediated gene transfer (Lavitrano et al., 1989 Cell 57:717-723). For a review of such techniques, see, Gordon, 1989 Transgenic Animals, Intl. Rev. Cytol. 115:171-129, which is incorporated by reference herein in its entirety. In one method, the canine MC5R nucleic acid is stably transfected into the MC5R-deficient mice disclosed in Chen et al., 1997 Cell 91:789-798.

The present invention provides for a transgenic animal that carries the canine MC5R transgene in all of its cells, as well as an animal which carries the transgene in some, but not all, of its cells, i.e., mosaic animals. The transgene may be integrated as a single transgene or in a concatamer, e.g., head-to-head or head-to-tail tandem repeats. The transgene may also be selectively introduced into and activated in a particular cell type by following, for example, the teaching of Lasko et al., 1992, Proc. Natl. Acad. Sci. USA 89: 6232-6236. The regulatory sequences required for such a cell-type specific activation will depend upon the particular cell type of interest, and will be apparent to those of skill in the art. When it is desired that the MC5R transgene be integrated into the chromosomal site of the endogenous MC5R gene, gene targeting is preferred. Briefly, when such a technique is to be utilized, a vector containing nucleic acids with sequences having a high percentage of identical nucleotide residues to the endogenous MC5R gene and/or sequences flanking the gene are designed for the purpose of integrating, via homologous recombination with chromosomal sequences, into and disrupting the function of the endogenous MC5R gene. The transgene also may be selectively expressed in a particular cell type with concomitant inactivation of the endogenous MC5R gene in only that cell type, by following, for example, the teaching of Gu et al., 1994 Science 265:103-06. The regulatory sequences required for such a cell-type specific recombination will depend upon the particular cell type of interest, and will be apparent to those of skill in the art.

Once a founder animal has been generated, standard techniques such as Southern blot analysis or PCR techniques are used to analyze animal tissues to determine whether integration of the transgene has taken place. The level of mRNA expression of the transgene in the tissues of the founder animal also may be assessed using techniques which include, but are not limited to, Northern blot analysis of tissue samples obtained from the animal, in situ hybridization analysis, and RT-PCR. Samples of MC5R gene-expressing tissue may also be evaluated immunocytochemically using antibodies specific for the MC5R transgene product.

A compound identified by an assay described above that stimulates or enhances the signal transduced by activated MC5R, e.g., by activating downstream signaling proteins in the MC5R cascade and thereby by-passing the defective MC5R, may be used to achieve weight loss or ameliorate skin conditions, as described below. The formulation and mode of administration will depend upon the physico-chemical properties of the compound. The administration should include known techniques that allow for a crossing of the blood-brain barrier.

Sources for Compounds Modulating the Activity of the MC5R of the Invention

The compounds that may be tested using the assays described above for MC5R agonist or antagonist activity include, but are not limited to, peptides such as, for example, soluble peptides, including but not limited to members of random peptide libraries; (see, e.g., Lam et al. 1991, Nature 354:82-84; Houghten et al., 1991 Nature 354:84-86), and combinatorial chemistry-derived molecular library made of D- and/or L-configuration amino acids, phosphopeptides (including, but not limited to, members of random or partially degenerate, directed phosphopeptide libraries (see, e.g., Songyang et al., 1993 Cell 72:767-78), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), the ECD of the MC5R (or a portion thereof) and bind to and “neutralize” natural ligand, and small organic or inorganic molecules.

Other compounds that can be screened in accordance with the invention include but are not limited to small organic molecules that are able to cross the blood-brain barrier, gain entry into an appropriate cell and affect the expression of the MC5R gene or some other gene involved in the MC5R signal transduction pathway (e.g., by interacting with the regulatory region or transcription factors involved in gene expression); or such compounds that affect the activity of the MC5R or the activity of some other intracellular factor involved in the MC5R signal transduction pathway, such as, for example, the MC5R associated G protein.

Identification of Ligands Using Computer Modeling

Computer modeling and searching technologies permit identification of a ligand, or the improvement of an already identified ligand, that can modulate MC5R expression or activity. Having identified the ligand, its active site or region is identified. The active site might typically be the MC5R binding site. The active site can be identified using methods known in the art including, for example, by examination of the amino acid sequence if the ligand is a peptide, from its nucleotide sequence if it is a nucleic acid, or from study of complexes of the compound or composition with MC5R. In the latter case, chemical or X-ray crystallographic examination of the complex can be used to find the active site by finding where the ligand and MC5R contact each other.

Next, the three dimensional geometric structure of the active site of the ligand is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric information. The geometry of the active site may be measured while the ligand is complexed with MC5R, or with another binding partner, which may increase the accuracy of the measurements.

If an incomplete or insufficiently accurate molecular structure of the ligand's active site is determined, the methods of computer based numerical modeling may be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site of the ligand, candidate ligands can be identified by searching databases containing compounds and information about their molecular structure. The search seeks compounds having structures that are identical or similar to the active site structure of the ligand. Such a search can be manual, but is preferably computer assisted. A compound identified in this search is a potential MC5R agonist or antagonist.

Alternatively, these methods can be used to create an improved agonist or antagonist from one that is already known. The composition of the known agonist or antagonist is modified and the structural affects of modification are determined using the experimental and computer modeling methods described above. Binding of the modified agonist or antagonist to MC5R, or its effect on MC5R-dependent signaling, is then compared to that of the original agonist or antagonist. Using these methods systematic variations of the known agonist or antagonist (e.g., systematic variation of a particular side group), can be quickly evaluated to obtain modified agonists or antagonists of improved specificity or activity. One or a plurality of these steps may be repeated serially to identify or create increasingly effective MC5R agonists or antagonists.

Further experimental and computer modeling methods useful for identifying an MC5R agonist or antagonist based upon identification of the active site of MC5R, and the active sites of related transduction and transcription factors, will be apparent to those of skill in the art.

Examples of molecular modeling systems are the CHARMm™ and QUANTA™ programs (Polygen Corporation, Waltham, Mass.). CHARMm™ performs the energy minimization and molecular dynamics functions. QUANTA™ performs the construction, graphic modeling and analysis of molecular structure. QUANTA™ allows interactive construction, modification, visualization, and analysis of the behavior of molecules with each other.

A number of articles review computer modeling of drugs interactive with specific proteins, such as Rotivinen, et al., 1988 Acta Pharmaceutical Fennica 97:159-166; Ripka, 1988 New Scientist 118:54-57; McKinaly and Rossmann, 1989 Annu. Rev. Pharmacol. Toxiciol. 29:111-122; Perry and Davies, 1989 OSAR: Quantitative Structure-Activity Relationships in Drug Design pp. 189-193 Alan R. Liss, Inc.; Lewis and Dean 1989 Proc. R. Soc. Lond. 236:125-140 and 141-162; and, with respect to a model receptor for nucleic acid components, Askew, et al., 1989 J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario, Canada). Although these are primarily designed for application to drugs specific to particular proteins, they can be adapted to design of drugs specific to regions of DNA or RNA, once that region is identified.

MC5R Peptides

In another aspect of the invention, canine MC5R protein, polypeptides and peptide fragments, mutated, truncated or deleted forms of the MC5R and/or MC5R fusion proteins are prepared for a variety of uses, including but not limited to the generation of antibodies, as reagents in diagnostic assays, the identification of other cellular gene products involved in the regulation of appetite and skin gloss in animals, as reagents in assays for screening for compounds that can be used in the treatment of appetite or skin disorders in animals, and as pharmaceutical reagents related to the MC5R useful in the treatment of appetite and skin disorders in animals.

A peptide corresponding to one or more domains of the MC5R (e.g., ECDs, TMs or ICs), a truncated or deleted MC5R (e.g., MC5R in which one or more of the ECDs, TMs and/or ICs is deleted) as well as a fusion protein in which the full length MC5R, an MC5R peptide or truncated MC5R is fused to an unrelated protein are also within the scope of the invention. Such a soluble peptide, protein, fusion protein, or antibody (including an anti-idiotypic antibody) that binds to and “neutralizes” circulating natural ligand for the MC5R can be used as described below to effectuate an increase in appetite. To this end, a peptide corresponding to an individual ECD of MC5R, a soluble deletion mutant of MC5R (e.g., ΔTM mutants), or the entire MC5R ECD (engineered by linking the four ECDs together as described below) can be fused to another polypeptide (e.g., an IgFc polypeptide). Fusion of the MC5R or the MC5R ECD to an IgFc polypeptide should not only increase the stability of the preparation, but will increase the half-life and activity of the MC5R-Ig fusion protein in vivo. The Fc region of the Ig portion of the fusion protein may be further modified to reduce immunoglobulin effector function.

Such a peptide, polypeptide, or fusion protein can be prepared by recombinant DNA techniques. For example, a nucleic acid encoding one or more of the four domains of the ECD of the serpentine MC5R can be synthesized or cloned and ligated together to encode a soluble ECD of the MC5R. Two or more nucleic acids, each encoding one or more of the four ECDs (ECD1-4 in FIG. 4), may be ligated together directly or via a linker oligonucleotide that encodes a peptide spacer. The linker may encode a flexible, glycine-rich polypeptide thereby allowing the ECDs that are strung together to assume a conformation that can bind an MC5R ligand. Alternatively, a nucleic acid encoding an individual domain within the ECD can be used to express an MC5R-derived peptide.

A variety of host-expression vector systems may be utilized to express a nucleic acid encoding the appropriate regions of the MC5R to produce the polypeptides described above. Where the resulting peptide or polypeptide is a soluble derivative (e.g., a peptide corresponding to an ECD; a truncated or internally-deleted MC5R) the peptide or polypeptide can be recovered from the culture medium. Where the polypeptide or protein is not secreted, the MC5R product can be recovered from the host cell itself.

The host-expression vector systems also encompass engineered host cells that express the MC5R or functional equivalents in situ, i.e., anchored in the cell membrane. Purification or enrichment of the MC5R from such expression systems can be accomplished using appropriate detergents and lipid micelles and methods well known to those skilled in the art. However, such engineered host cells themselves may be used in situations where it is important not only to retain the structural and functional characteristics of the MC5R, but to assess biological activity, e.g., in drug screening assays, see, supra.

A fusion protein may be readily purified by utilizing an antibody specific for the fusion protein being expressed. For example, one such system allows for the ready purification of non-denatured fusion proteins expressed in human cell lines (see, Janknecht, et al., 1991 Proc. Natl. Acad. Sci. USA 88: 8972-76). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺-nitriloacetic acid-agarose columns and histidine-tagged proteins are selectively eluted with imidazole-containing buffers.

MC5R Antibodies

In again another aspect, antibodies that specifically recognize one or more epitopes of canine MC5R, or epitopes of conserved variants of MC5R, or peptide fragments of the MC5R are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

An antibody of the invention may be used, for example, in the detection of MC5R in a biological sample and may, therefore, be utilized as part of a diagnostic or prognostic technique whereby an animal may be tested for an abnormal amount of MC5R. The antibody also may be utilized in conjunction with, for example, a compound screening scheme, as described, above, for the evaluation of the effect of a test compound on expression and/or activity of the MC5R gene product. Additionally, the antibody may be used in conjunction with the transgenic techniques described, below, e.g., to evaluate the normal and/or engineered MC5R-expressing cells prior to their introduction into the animal subject. The antibody additionally may be used as a method for the inhibition of abnormal MC5R activity. Thus, the antibody may be utilized as part of an appetite disorder or skin disorder treatment method.

For the production of the antibody, a host animal may be immunized by injection with MC5R, an MC5R peptide (e.g., one corresponding the a functional domain of the receptor, such as ECD, TM or IC), a truncated MC5R polypeptide (i.e., MC5R in which one or more domains, e.g., the TM or IC, has been deleted), a functional equivalent of the MC5R or a mutant of the MC5R. The host animal may be, e.g., a goat, rabbit, mouse, hamster or rat. An adjuvant may be used to increase the immunological response, depending on the host species. The adjuvant may be, e.g., Freund's (complete and incomplete), mineral gels (e.g. aluminum hydroxide), surface active substances such as lysolecithin, pluronic polyols, polyanions, peptides, oil emulsions, keyhole limpet hemocyanin, dinitrophenol, and potentially useful human adjuvants such as bacille Calmette-Guerin (hereinafter “BCG”) and Corynebacterium parvum.

A polyclonal antibody is a heterogeneous population of antibody molecules derived from the serum of an immunized animal. A monoclonal antibody (hereinafter “mAb”) is a homogeneous population of antibodies. A mAb to a particular antigen may be generated by any technique that provides for the production of antibody molecules by a continuous cell line in culture. These techniques include, but are not limited to, the hybridoma technique of Kohler and Milstein, 1975 Nature 256:495-497; and U.S. Pat. No. 4,376,110, the human B-cell hybridoma technique (Kosbor et al., 1983 Immunology Today 4:72; Cole et al., 1983 Proc. Natl. Acad. Sci. USA 80:2026-2030), and the EBV-hybridoma technique (see, Cole et al., 1985 Monoclonal Antibodies And Cancer Therapy, Alan R. Liss, Inc., pp. 77-96). The antibody may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (see, Morrison et al., 1984 Proc. Natl. Acad. Sci. USA 81:6851-6855; Neuberger et al., 1984 Nature 312:604-608; Takeda et al. 1985, Nature 314:452-454) may be used. A chimeric antibody is a molecule comprising portions from different animal species, such as those having a variable region derived from a murine mAb and a human immunoglobulin constant region. A chimeric antibody may be generated, e.g., by splicing the genes from a mouse antibody molecule of appropriate antigen specificity together with genes from a human antibody molecule of appropriate biological activity.

Alternatively, techniques described for the production of a single chain antibody (see, U.S. Pat. No. 4,946,778; Bird, 1988 Science 242:423-26; Huston et al., 1988 Proc. Natl. Acad. Sci. USA 85:5879-83; and Ward et al., 1989 Nature 334:544-546) may be adapted to produce single chain antibodies against MC5R gene products. A single chain antibody is formed by linking the heavy and light chain fragments of the Fv region via an amino acid bridge, resulting in a single chain polypeptide.

An antibody fragment that recognizes a specific epitope may be generated by known techniques. For example, the antibody fragment may be, e.g., one of the F(ab′)₂ fragments that results from pepsin digestion of the antibody molecule, or one of the Fab fragments that is generated by reducing the disulfide bridges of the F(ab′)₂ fragments. Alternatively, a Fab expression library may be constructed (see, Huse et al., 1989 Science 246:1275-1281) to allow rapid and easy identification of a monoclonal Fab fragment with the desired specificity.

An antibody to the MC5R may, in turn, be utilized to generate an anti-idiotype antibody that “mimics” MC5R, using techniques well known to those skilled in the art (see, e.g., Greenspan and Bona, 1993 FASEB J. 7:437-444; and Nissinoff, 1991 J. Immunol. 147:2429-2438). For example, an antibody that binds to the MC5R ECD and competitively inhibits the binding of a melanocortin to MC5R may be used to generate an anti-idiotype that “mimics” the ECD and, therefore, binds to and neutralizes melanocortins. The neutralizing anti-idiotype (or Fab fragments of the anti-idiotype) can be used in a therapeutic regimen to neutralize the native ligand and promote increased appetite in a subject animal.

Alternatively, an antibody to MC5R that acts as an agonist of MC5R may be generated. The antibody binds to MC5R and activates its signal transducing activity. The antibody is particularly useful for treating appetite-related disorders such as obesity in a subject animal and for treating dry skin disorders. In addition, an antibody that acts as antagonist of MC5R, i.e. that inhibits the activation of MC5R receptor, may be used to treat appetite-related disorders such as anorexia or cachexia in a subject animal, or oily skin conditions such as seborrhea.

The Treatment of Disorders Using the Compounds Identified by the Methods of the Invention

The invention encompasses methods and compositions for modifying appetite and/or metabolic rate and treating appetite- and/or metabolic rate-related disorders in animals, including but not limited to obesity, cachexia, anorexia, reproductive incompetence, weaning-induced inappetance and growth lag, and lactation. The invention also encompasses methods and compositions for treating skin disorders such as seborrhea, psoriasis, and dermatitis; cardiovascular disease; endotoxemia, fever, renal failure, hepatic lipidosis, cancer; infection; inflammation and hypertension. An increase in MC5R activity, or activation of the MC5R pathway (e.g., downstream activation) would facilitate progress towards a normal body weight state in obese animals exhibiting a deficient level of MC5R gene expression and/or MC5R activity. Such an increase would also facilitate an increase in sebaceous gland excretion, thus ameliorating dry skin conditions in animals exhibiting a deficient level of MC5R gene expression and/or activity. Alternatively, symptoms of certain disorders such as, for example, cachexia, which include inappetence and perhaps a lower than normal body weight phenotype, and skin disorders characterized by excessive sebaceous gland secretion such as seborrhea, may be ameliorated by decreasing the level of MC5R gene expression, and/or MC5R gene activity, and/or downregulating activity of the MC5R pathway (e.g., by targeting downstream signaling events). Different approaches are discussed below.

In one embodiment, a compound that modulates MC5R activity is administered to a subject animal, preferably a mammal or a bird, in need thereof. The subject animal may suffer from one of the diseases or conditions listed above. The compound may be an agonist or an antagonist of MC5R. An agonist of MC5R may be used to reduce food intake and/or increase metabolic rate to induce weight loss for treating obesity in a subject animal. In a preferred embodiment, a preparation comprising an MC5R agonist that induces safe, effective appetite reduction is administered to an animal in need thereof. In particularly preferred embodiments, the animal is a dog, the weight loss is between 4-8% of the excess weight of the animal per month, and/or the preparation is administered orally. An antagonist of MC5R activity may be used to induce increased appetite for treating conditions such as anorexia or cachexia, or to reduce sebaceous gland excretion, particularly of sebum excretion, to treat conditions such as seborrhea. In a preferred embodiment, a preparation comprising an MC5R antagonist that acutely stimulates the appetite is administered to an animal in need thereof. In a particularly preferred embodiment, the animal is a dog suffering from pathology that results in inappropriately low food intake and weight loss (e.g., hepatic lipidosis or cachexia), or that results in excessive sebum production (e.g., seborrhea).

It is not necessary that the compound demonstrate absolute specificity for the MC5R. For example, a compound that agonizes both MC5R and MC1R could be used; the compound could be administered so that delivery to the brain is optimized to achieve reduced appetite and weight reduction, and side effects, such as peripheral melanin production resulting in the skin, hide or fur, are reduced to a tolerable level. A compound that does not demonstrate a specificity for MC5R may be administered in conjunction with another therapy or drug to control the side-effects that may result from modulating another melanocortin receptor.

Toxicity and therapeutic efficacy of the compound can be determined by standard pharmaceutical procedures in cell cultures or experimental animals, such as rats and mice, e.g., for determining the LD₅₀ (the dose lethal to 50% of the population) and the ED₅₀ (the dose therapeutically effective in 50% of the population). The dose ratio between toxic and therapeutic effects (i.e. the ratio LD₅₀/ED₅₀) is the therapeutic index. A compound that exhibits a large therapeutic index is preferred. While a compound that exhibits toxic side effects may be used, care should be taken to design a delivery system that targets the compound to the site of affected tissue in order to minimize potential damage to uninfected cells and, thereby, reduce side effects.

The data obtained from the cell culture assays and experimental animal studies for a compound may be used in formulating a dosage range for use of the compound in a subject animal, such as a cat, dog or livestock. The dosage of the compound lies preferably within a range of circulating concentrations that include the ED₅₀ with little or no toxicity. The dosage may vary within this range depending upon the dosage form employed and the route of administration utilized. The therapeutically effective dose of the compound may be estimated initially from cell culture assays. A dose may be formulated in experimental animal models to achieve a circulating plasma concentration range that includes the IC₅₀ (i.e., the concentration of the test compound which achieves a half-maximal inhibition of symptoms) as determined in cell culture. Such information may be used to more accurately determine a useful dose in a subject animal. Levels of the compound in plasma may be measured, for example, by high performance liquid chromatography.

Antisense Oligonucleotides Used for Inhibiting the Expression of MC5R

In yet another aspect of the invention, a nucleic acid molecule is used to inhibit the expression of a component of the MC5R signal transduction pathway, thereby modulating the appetite of a subject animal. In one embodiment, a therapy is designed wherein the level of endogenous MC5R gene expression in the subject animal is reduced by using an antisense nucleic acid or a ribozyme to inhibit or prevent translation of MC5R mRNA transcripts; a nucleic acid that forms a triple helix with all or part of the MC5R gene or its regulatory elements to inhibit transcription of the MC5R gene; or a nucleic acid useful for targeted homologous recombination to inactivate or “knock out” the MC5R gene or its endogenous promoter. The therapy may be utilized for treatment of appetite and body weight disorders in the animal subject such as cachexia and anorexia where the inhibition of MC5R expression is designed to increase appetite, or for treatment of exocrine gland disorders such as seborrhea where the inhibition of MC5R expression is designed to reduce gland secretions. Because it is believed that MC5R functions to regulate appetite through its activity in the brain, delivery techniques preferably should be designed to allow the nucleic acid to cross the blood-brain barrier (see, PCT WO89/10134, which is incorporated by reference herein in its entirety). For example, the nucleic acids can be modified, or appropriately formulated, to increase their ability to cross the blood-brain barrier. Alternatively, the antisense, ribozyme or nucleic acid constructs described herein could be administered directly to the site containing the target cells.

An antisense approach involves the design of an oligonucleotide that is complementary to an mRNA. The oligonucleotide may comprise any suitable polymeric molecule, e.g., DNA, RNA, a DNA/RNA hybrid, modified DNA or RNA, or a synthetic DNA or RNA analog (e.g., peptide nucleic acid (hereinafter “PNA”); see, WO 92/20702), as described more fully below. The antisense oligonucleotide will bind to the complementary mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of a double-stranded antisense nucleic acid, a single strand of the duplex DNA thus may be tested, or triplex formation may be assayed. The ability to hybridize will depend on both the degree of complementarity and the length of the antisense oligonucleotide. Generally, the longer the hybridizing oligonucleotide, the more base mismatches with the RNA it may contain and still form a stable duplex (or triplex, as the case may be). One skilled in the art can ascertain a tolerable degree of mismatch by use of standard procedures to determine the melting point of the hybridized complex.

While antisense oligonucleotides complementary to the coding region sequence may be used, those complementary to the transcribed untranslated region are most preferred. Oligonucleotides that are complementary to the 5′ end of the message, e.g., the 5′ untranslated region up to and including the AUG initiation codon, should work most efficiently at inhibiting translation (see FIG. 1). However, sequences complementary to the 3′ untranslated sequences of mRNAs have proven to be effective at inhibiting translation of mRNA as well. See, generally, Wagner, 1994 Nature 372:333-335. Thus, an oligonucleotide complementary to either the 5′- or 3′-non-translated, non-coding region of MC5R may be used in an antisense approach to inhibit translation of endogenous mRNA. An oligonucleotide complementary to the 5′ untranslated region of the mRNA preferably includes the complement of the AUG start codon. An antisense oligonucleotide complementary to mRNA coding regions is a less efficient inhibitor of translation but may be used in accordance with the invention. Whether designed to hybridize to the 5′-, 3′- or coding region of MC5R mRNA, an antisense oligonucleotide should be at least six nucleobases in length, and preferably ranging from 6 to about 50 nucleobases in length. A nucleobase is a monomer unit from which the oligonucleotide is comprised (e.g., for DNA and RNA oligonucleotides, a nucleobase is a nucleotide). In specific aspects the oligonucleotide is at least 10 nucleobases, at least 17 nucleobases, at least 25 nucleobases or at least 50 nucleobases.

Ribozymes

A ribozyme molecule designed to catalytically cleave MC5R mRNA transcripts also may be used to prevent translation of MC5R mRNA and expression of MC5R in a subject animal (see, e.g., PCT International Publication WO90/11364, published Oct. 4, 1990; Sarver et al., 1990 Science 247:1222-1225). While a ribozyme that cleaves mRNA at site specific recognition sequences can be used to destroy MC5R mRNAs, the use of a hammerhead ribozyme is preferred. A hammerhead ribozyme cleaves mRNAs at locations dictated by flanking regions that form complementary base pairs with the target mRNA. The sole requirement is that the target mRNA have the following sequence of two bases: 5′-UG-3′. The construction and production of a hammerhead ribozyme is well known in the art and is described more fully in Haseloff and Gerlach, 1988 Nature 334:585-591. There are hundreds of potential hammerhead ribozyme cleavage sites within the nucleotide sequences of canine MC5R cDNA (see FIG. 3). Preferably the ribozyme is engineered so that the cleavage recognition site is located near the 5′ end of the MC5R mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (see, Zaug at al., 1984 Science 224:574-578; Zaug and Cech, 1986 Science 231:470-475; Zaug at al., 1986 Nature 324:429-433; published International patent application No. WO 88/04300 by University Patents Inc.; Been and Cech, 1986 Cell 47:207-216). A Cech-type ribozyme has an eight base pair active site which hybridizes to a target RNA sequence whereafter cleavage of the target RNA takes place. The invention encompasses those Cech-type ribozymes which target eight base-pair active site sequences that are present in MC5R.

As in the antisense approach, the ribozymes can be composed of modified oligonucleotides (e.g. for improved stability, targeting, etc.) and should be delivered to cells which express the MC5R in vivo, e.g., skin. For example, the ribozyme can be modified, or appropriately formulated, to cross the blood-brain barrier (see, PCT WO89/10134, which is incorporated by reference herein in its entirety). A preferred method of delivery involves using a DNA construct encoding the ribozyme under the control of a strong constitutive pol III or pol II promoter, so that transfected cells will produce sufficient quantities of the ribozyme to destroy endogenous MC5R messages and inhibit translation. Because ribozymes, unlike antisense molecules, are catalytic, a lower intracellular concentration is required for efficiency.

Transgenic Animals

In another aspect of the invention, transgenic animals are generated that exhibit altered appetite regulation and body weight control and/or skin or coat lipid levels compared to their wild type counterparts. Specifically, in such animals, the expression of MC5R is controlled in vivo, e.g., at the transcriptional or translational level. Preferably, the transgenic animal is a non-human mammal, e.g., a dog, a cat, a cow, a horse, a sheep, a goat, a mouse, a rat or a pig. Certain approaches are described below.

With respect to an increase in the level of normal MC5R gene expression and/or MC5R gene product activity, canine MC5R nucleic acid sequences can be utilized to generate transgenic animals less prone to suffer from appetite and body weight disorders, including obesity, and skin disorders.

Alternatively, targeted homologous recombination can be utilized to correct a defective endogenous MC5R gene in the appropriate tissue of an animal subject; e.g., brain or skin tissue. Targeted homologous recombination can be used to correct the defect in embryonic stem cells (“ES cells”) in order to generate offspring with a corrected trait.

In another alternative, endogenous MC5R gene expression in an animal subject can be reduced by inactivating or “knocking out” the MC5R gene or its promoter using targeted homologous recombination. Smithies et al., 1985 Nature 317:230-234; Thomas and Capecchi, 1987 Cell 51:503-512; Thompson et al., 1989 Cell 5:313-321; each of which is incorporated by reference herein in its entirety. For example, a mutant, non-functional MC5R (or a completely unrelated DNA sequence) flanked by DNA homologous to the endogenous MC5R gene can be used, with or without a selectable marker and/or a negative selectable marker, to transfect cells that express MC5R in vivo. Insertion of the DNA construct, via targeted homologous recombination, results in inactivation of the MC5R gene. This approach is particularly suited for agricultural applications where modifications to ES cells can be used to generate animal offspring with an inactive MC5R. See, e.g., Thomas and Capecchi and Thompson, 1987, supra. However this approach may be adapted for use in companion animals such as dogs.

An MC5R deficient mouse is disclosed in Chen et. al., 1997, supra. Such a “knock out” mouse can be further modified to express the canine MC5R by “knocking in” the canine gene to produce a “caninized” mouse. Preferably, such a mouse would overexpress canine MC5R. Such a mouse would be a useful animal model for screening compounds for activity at the canine MC5R.

Alternatively, endogenous MC5R gene expression in an animal subject may be reduced by targeting deoxyribonucleotide sequences complementary to the regulatory region of the MC5R gene (i.e., the MC5R promoter and/or enhancers) to form triple helical structures that prevent transcription of the MC5R gene in target cells in the body. See, generally Helene, 1991 Anticancer Drug Des. 6:569-584; Helene et al., 1992 Ann. N.Y. Acad. Sci. 660:27-36; and Maher, 1992 Bioassays 14:807-815.

Genetically engineered cells that express soluble MC5R ECDs or fusion proteins, e.g. fusion Ig molecules, may be administered in vivo where they may function as “bioreactors” that deliver a supply of the soluble molecules. Such soluble MC5R polypeptides and fusion proteins, when expressed at appropriate concentrations, should neutralize or “mop up” the native ligand for MC5R, and thus act as inhibitors of MC5R activity and induce appetite and perhaps weight gain in the subject animal.

Pharmaceutical Formulations and Methods of Administration

A pharmaceutical composition comprising the MC5R agonists and antagonists of the invention for use in accordance with the present invention may be formulated in conventional manner. The pharmaceutical composition comprises a compound that modulates MC5R activity (as described above) and one or more physiologically acceptable carriers or excipients. The carriers or excipients are selected according to the method of administration to be used. The compound (and its physiologically acceptable salts and solvates) may be formulated, e.g., for administration by inhalation or insufflation (either through the mouth or the nose) or oral, buccal, parenteral, topical, transdermal or rectal administration.

For oral administration, the pharmaceutical composition may take the form of, for example, tablets or capsules prepared by conventional means with pharmaceutically acceptable excipients such as a binding agent (e.g., pregelatinised maize starch, polyvinylpyrrolidone or hydroxypropyl methylcellulose); a filler (e.g., lactose, microcrystalline cellulose or calcium hydrogen phosphate); a lubricant (e.g., magnesium stearate, talc or silica); a disintegrant (e.g., potato starch or sodium starch glycolate); or a wetting agent (e.g., sodium lauryl sulphate). The tablets may be coated by methods well known in the art. A liquid preparation for oral administration may take the form of, for example, a solution, syrup or suspension, or it may be presented as a dry product for constitution with water or other suitable vehicle before use. The liquid preparation may be prepared by conventional means with a pharmaceutically acceptable additive such as a suspending agent (e.g., sorbitol syrup, cellulose derivatives or hydrogenated edible fats); emulsifying agent (e.g., lecithin or acacia); non-aqueous vehicle (e.g., almond oil, oily esters, ethyl alcohol or fractionated vegetable oils); or preservative (e.g., methyl or propyl-p-hydroxybenzoates or sorbic acid). The preparation also may contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compound is conveniently delivered in the form of an aerosol spray presentation from a pressurized pack or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compound may be formulated for parenteral administration by injection, e.g., by bolus injection or continuous infusion. A formulation for injection may be presented in unit dosage form, e.g., in ampoules or in multi-dose containers, with an added preservative. The formulation may take the form of a suspension, solution or emulsion in an oily or aqueous vehicle, and may contain formulatory agents such as suspending, stabilizing and/or dispersing agents. Alternatively, the compound may be in powder form for constitution with a suitable vehicle, e.g., sterile pyrogen-free water, before use.

The compound may also be formulated in a rectal composition such as a suppository or retention enema, e.g., containing a conventional suppository base such as cocoa butter or other glyceride.

In addition to the formulations described previously, the compound also may be formulated as a long-acting depot preparation. The preparation may be administered by implantation (e.g., subcutaneously or intramuscularly), by intramuscular injection or by a transdermal patch. Thus, e.g., the compound may be formulated with a suitable polymeric or hydrophobic material (e.g., as an emulsion in an acceptable oil) or ion exchange resin, or as a sparingly soluble derivative, e.g., as a sparingly soluble salt.

The composition may, if desired, be presented in a pack or dispenser device that may contain one or more unit dosage forms containing the active ingredient. The pack may comprise, e.g., metal or plastic foil, such as a blister pack. The pack or dispenser device may be accompanied by instructions for administration.

In another aspect of the invention, a system described above may be formulated into a kit. To this end, the MC5R or cells expressing the MC5R can be packaged in a variety of containers, e.g., vials, tubes, microtiter well plates, bottles, and the like. Other reagents can be included in separate containers and provided with the kit; e.g., positive controls samples, negative control samples, melanocortin peptides (including but not limited to α-MSH and ACTH derivatives), buffers, cell culture media, etc.

### EXAMPLE 1

The following example describes the isolation of the nucleic acids encoding the canine melanocortin receptor polypeptides disclosed herein.

Canine skin tissues are prepared. cDNA libraries are prepared therefrom by Life Technologies, Inc. (Rockville, Md.). A biotinylated capture oligonucleotide having the sequence CATAGGGGCCATAGTGAAGAACAAAAACC is designed from a conserved region of the MC5R polynucleotide sequence. Canine MC5R clones are isolated from their corresponding cDNA libraries using this oligonucleotide and the GENETRAPPER® system (Life Technologies, Inc.). Positive selection is achieved by stringently hybridizing this oligonucleotide to clones in a cDNA library. The complex is separated from all other clones in the library using Streptaviden magnetic beads, which are pelleted using a magnet. MC5R clones are identified by PCR using a MC5R specific primer pair designed to be outside of the capture oligonucleotide site (forward primer: TTCGCCGTGTGAAGAGATGG; reverse primer: ACGCCTCACGGTCATGATGT). One canine MC5R clone is isolated from the cDNA libraries, yielding a clone of 3.9 kilobases in size. Identity of the clones is confirmed by PCR using a different MC5R polynucleotide sequence-specific primer pair (forward primer: TGAGGCGTTCCAGAGTGTTAT; reverse primer: GGAGCCCAGCACACGAT).

Sequencing of the canine MC5R polynucleotide is completed using one standard and seven primer walking reactions from both ends of each clone. Sequences of the MC5R polynucleotide fragments are assembled based on their overlapping regions.

The nucleotide sequence encoding canine MC5R is depicted in SEQ ID NO:1. Conceptual translation of the open reading frame (ORF) of the nucleotide sequence revealed the predicted amino acid sequence of canine MC5R, shown in SEQ ID NO:2. The open reading frame nucleotide sequence is depicted in SEQ ID NO:3.

The canine MC5R nucleotide ORF sequence is compared with other available MC5R polynucleotide sequence sequences from other species. The canine MC5R nucleotide sequence is used as a query to search the public sequence databases. The ORF and protein sequence identities between the queries and the hits are analyzed using the Jotun Hein method of the LASERGENE-MEGALIGN™ software package (DNASTAR, Inc., Madison, Wis.) to determine divergence. The Jotun Hein method builds a phylogenetic tree by examining sequence pairs and creating the best possible arrangement of ancestral branches. The method is most useful when aligned sequences are related by descent. The following TABLES I and II show the result of the query.

TABLE I
Percentage identities of MC5R nucleotide sequences
(open reading frame) among different species

| Species | Canine | human | Chimpanzee | bovine | Mouse | rat |
|---|---|---|---|---|---|---|
| Canine | 100 | 85.4 | 86.2 | 79.9 | 83.2 | 83.7 |
| Human | | 100 | 99.2 | 81.9 | 81.4 | 82.7 |
| chimp- | | | 100 | 82 | 83 | 83 |
| anzee | | | | | | |
| Bovine | | | | 100 | 78.5 | 79.2 |
| Mouse | | | | | 100 | 91.7 |
| Rat | | | | | | 100 |

TABLE II
Percentage identities of MC5R amino acid
sequences among different species

| Species | Canine | human | chimpanzee | bovine | Mouse | rat |
|---|---|---|---|---|---|---|
| Canine | 100 | 86.2 | 86.7 | 79.2 | 83.2 | 85.0 |
| Human | | 100 | 99.4 | 79.8 | 80.4 | 83.1 |
| Chimp- | | | 100 | 76 | 83 | 94 |
| anzee | | | | | | |
| Bovine | | | | 100 | 75.8 | 75.5 |
| Mouse | | | | | 100 | 94.5 |
| Rat | | | | | | 100 |

### EXAMPLE 2

The following example illustrates the ability of canine MC5R and MC5R to bind α-MSH.

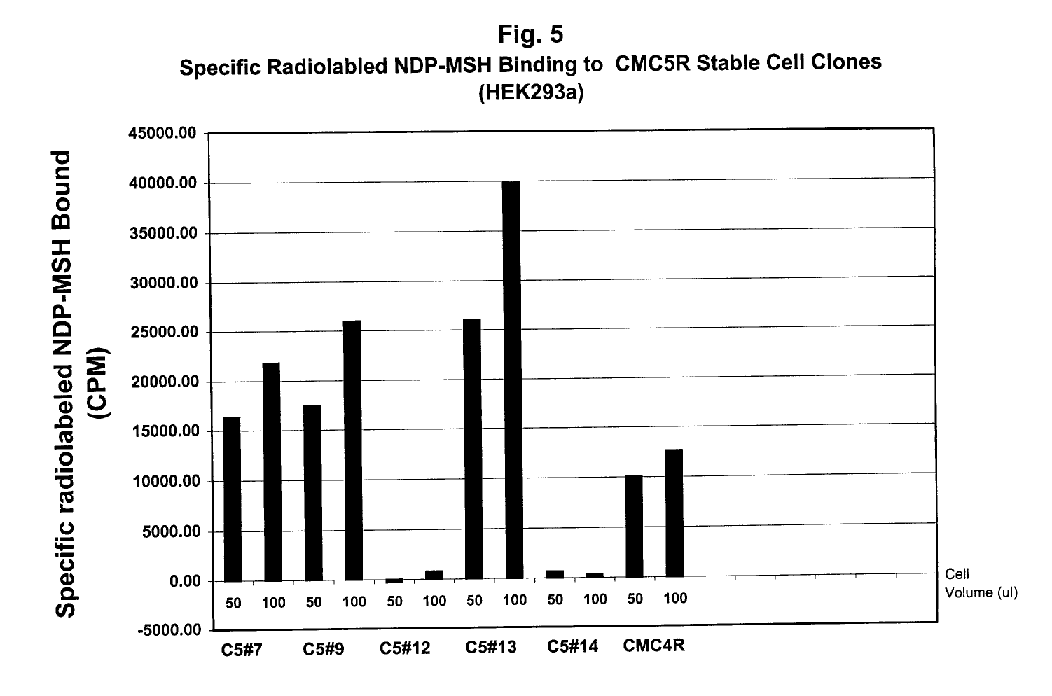

Transient transfection & Whole Cell Binding Assay. A 50% confluent cell culture plate containing HEK293 cells is used for transient transfection with various vector constructs using Fugene 6 as a transfection carrier (4 μl Fugene 6 per μg plasmid DNA). Cells are harvested 48 hours post transfection using Sigma dissociation buffer (Sigma Chemical Co., St. Louis, Mo.), then centrifuged and resuspended in binding buffer (50 mM HEPES, 5 mM MgCl, 0.1% BSA, pH 7.5, containing a Sigma P-8340 protease inhibitor cocktail). The resuspended cell population is counted using a hemacytometer. A cell volume titration is performed by pipetting varying volumes in triplicate into tubes. Either buffer or excess cold NDP-MSH (2 μM final conc.) is added. Excess cold NDP-MSH is used to ascertain non-specific binding. Radiolabeled (¹²⁵I) NDP-MSH is added such that the final concentration is 75 pM. The reaction is incubated at 37° C. for 1 hour, and then the reaction mixture is centrifuged at 5000×g for 10 minutes. The supernatant solution is removed by aspiration and radioisotope remaining with the cell pellet is counted in a gamma counter. FIG. 5 discloses the specific NDP-MSH binding to five canine MC5R/HEK293a cells (shown as “C5#_” in FIG. 5). The sixth set (CMC4R) in FIG. 5 shows canine MC4R as a positive control. Clones nos. 9 and 13 are notable for the high level of specific binding.

Membrane binding assay. HEK293 cells transfected with MCR cDNA are harvested using Sigma dissociation buffer, then centrifuged and resuspended in ice cold homogenization buffer (1 mM EDTA, 1 mM EGTA, 10 mM HEPES, pH 7.5, containing Sigma P-8340 protease inhibitor cocktail). The cell suspension is incubated at 4° C. for at least 10 minutes. The cell suspension is homogenized with 20 strokes of a tight fitting glass/glass dounce homogenizer. The homogenate is centrifuged at 1000×g for 10 minutes at 4° C. to pellet nuclei and unlysed cells. Then the supernatant solution is transferred to a new tube and centrifuged at 25000×g for 20 minutes at 4° C. in order to pellet the plasma membrane. The supernatant solution is discarded and the pellet resuspended in homogenization buffer in order to wash the plasma membrane. The pellet is homogenized with 20 strokes of a tight fitting glass/glass dounce homogenizer and the suspension is then centrifuged at 25000×g for 20 minutes at 4° C. The resultant supernatant solution is discarded and the pellet resuspended in an appropriate volume of homogenization buffer that will yield a protein concentration of 1-5 mg/ml. The resuspended pellet is divided into aliquots of 500 μl and frozen at −70° C. for long term storage. The protein concentration is measured by use of a Pierce BCA kit (Pierce Chemical Co., Rockford, Ill.).

A canine MC5 receptor-binding assay is performed using membranes isolated as described above. Radiolabeled NDP-MSH (NEN Life Science Products, Boston, Mass.) is used at a final concentration of 0.050 nM. Cell membranes of canine MC5R/HEK293 and an unlabeled test compound are resuspended in assay buffer (50 mM HEPES, 5 mM MgCl, 0.1% BSA, pH 7.5, containing a Sigma P-8340 protease inhibitor cocktail) and incubated at 37° C. for 1.3 hours. The binding reaction is terminated by harvesting membranes onto PEI treated GFC filter plates. Scintillant is added and the bound radioisotope is measured with a beta counting instrument. All assays are performed in a 96 well plate format. Unlabeled test compounds are examined over a range of concentrations from 0.01 nM to 20,000 nM and IC₅₀ values are determined.

### EXAMPLE 3

The following example discloses the measurement of canine MC5R-mediated cAMP accumulation in response to α-MSH stimulation.

As described above, MC5R, when activated by its agonist ligand, stimulates adenylyl cyclase to form cAMP. A commercially available kit (Adenylyl Cyclase Activation Flashplate Assay, NEN® Life Science Products, Inc., Boston, Mass., Cat # SMP004, SMP004A) is used to measure cAMP accumulation. An MC5R/HEK293 cell line is prepared. Cells are harvested using Sigma dissociation buffer, washed, centrifuged and resuspended in stimulation buffer, which is provided in the kit (Sigma Chemical Co., St. Louis, Mo.). The suspended cells are counted using a hemacytometer. Cells are pelleted again by centrifugation and resuspended with stimulation buffer to the desired concentration (0.5-5×10⁶ cells per ml). A cell volume titration is performed by transferring varying volumes in triplicate into wells. For each cell concentration tested, a full agonist dose titration is performed, including a negative control. The agonists employed include [Nle⁴,D-Phe⁷]-α-MSH(NDP-MSH) and α-MSH. These molecules can be purchased from commercial sources such as Sigma Chemical Co. or Bachem AG (Bubendorf, Switzerland).

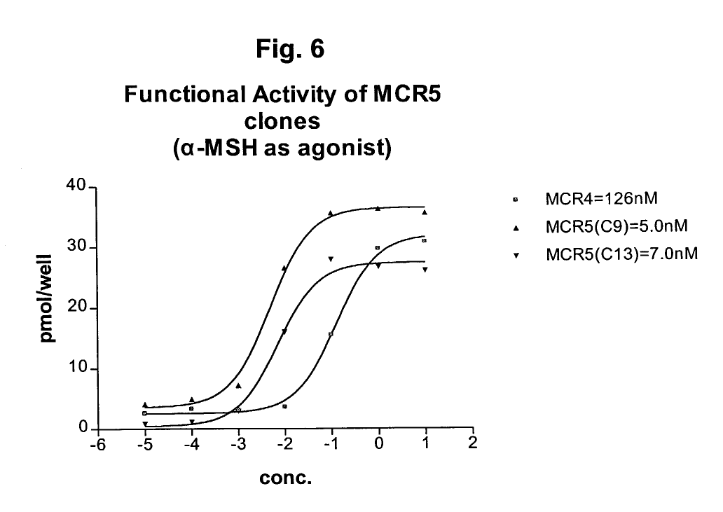

The duration of stimulation by agonist is also varied (15-45 minutes). Stimulation by agonist is terminated by adding detection mix (provided in the kit) at the appropriate time to all wells. Plates are covered and incubated for 20 hours at room temperature. Stimulation of MC5R is determined by scillintation counting in a Wallac MICROBETA® scintillation counter (EG&G Wallac, Gaithersburg, Md.). Other cAMP assay kits can be used such as the Amersham cAMP SPA kit (RPA559) (Amersham Corp., Arlington Heights, Ill.) or other kits by other vendors. The result of treatment of three clones with α-MSH is depicted in FIG. 6, where stimulation with α-MSH resulted in up to an 8-fold stimulation. The EC₅₀ for clone C9 is found to be 5 nM. Both MC5R clones are measured to be more sensitive to α-MSH than a MC4R clone.

1. An isolated nucleic acid encoding a functional MC5R, or the complement thereof, said nucleic acid comprising a nucleotide sequence selected from the group consisting of:
(a) a nucleotide sequence which hybridizes under conditions of moderate stringency to the coding region of SEQ ID NO:1;
(b) a nucleotide sequence which hybridizes under conditions of moderate stringency to a polynucleotide which is complementary to the coding region of SEQ ID NO:1;
(c) a nucleotide sequence which hybridizes under conditions of moderate stringency to the coding region of the canine MC5R as deposited with the ATCC and having ATCC Accession No. PTA-3455; and
(d) a nucleotide sequence which hybridizes under conditions of moderate stringency to a polynucleotide which is complementary to the coding region of the canine MC5R as deposited with the ATCC and having ATCC Accession No. PTA-3455, with the proviso that said functional MC5R is not human, bovine, chimpanzee, murine, rat, chicken or swine.

2. An isolated nucleic acid encoding a functional MC5R, or the complement thereof, said nucleic acid comprising a nucleotide sequence selected from the group consisting of:
(a) a nucleotide sequence which hybridizes under conditions of high stringency to the coding region of SEQ ID NO:1;
(b) a nucleotide sequence which hybridizes under conditions of high stringency to a polynucleotide which is complementary to the coding region of SEQ ID NO:1;
(c) a nucleotide sequence which hybridizes under conditions of high stringency to the coding region of the canine MC5R as deposited with the ATCC and having ATCC Accession No. PTA-3455; and
(d) a nucleotide sequence which hybridizes under conditions of high stringency to a polynucleotide which is complementary to the coding region of the canine MC5R as deposited with the ATCC and having ATCC Accession No. PTA-3455.

3. An isolated nucleic acid comprising a nucleotide sequence that:
(a) encodes a polypeptide according to SEQ ID NO:2; or
(b) encodes a polypeptide encoded by the canine MC5R clone as deposited with the ATCC and having ATCC Accession No. PTA-3455.

4. The isolated nucleic acid of claim 3 wherein said nucleic acid has a nucleotide sequence according to SEQ ID NO: 1, SEQ ID NO:3 or the canine MC5R clone as deposited with the ATCC and having ATCC Accession No. PTA-3455.

5. An isolated nucleic acid comprising a nucleotide sequence having more than about 87% identity to SEQ ID NO:3 or to the canine MC5R clone as deposited with the ATCC and having ATCC Accession No. PTA-3455.

6. An isolated nucleic acid comprising a nucleotide sequence encoding a polypeptide having more than about 87% identity to SEQ ID NO:2 or to the polypeptide encoded by the canine MC5R clone as deposited with the ATCC and having ATCC Accession No. PTA-3455.

7. An isolated nucleic acid comprising a nucleotide sequence encoding an extracellular domain of a canine MC5R corresponding to amino acids 1-37, 90-119, 181-185, or 265-272 of SEQ ID NO:2 or of the polypeptide encoded by the canine MC5R clone as deposited with the ATCC and having ATCC Accession No. PTA-3455.

8. An isolated nucleic acid comprising a nucleotide sequence encoding a cytoplasmic domain of a canine MC5R corresponding to amino acids 63-70, 139-160, 213-217, or 297-325 of SEQ ID NO:2 or of the polypeptide encoded by the canine MC54R clone as deposited with the ATCC and having ATCC Accession No. PTA-3455.

9. A nucleotide vector comprising the nucleic acid of claim 1 under the control of a nucleotide regulatory element with which the nucleic acid is not naturally associated.

10. A genetically engineered host cell that expresses functional MC5R and comprises the nucleic acid of claim 1.

11. The genetically engineered host cell of claim 10 wherein the nucleic acid is in operative association with a nucleotide regulatory element that controls expression of said nucleotide sequence in the host cell.

12. An isolated polypeptide encoded by the nucleic acid of claim 1.

13. An isolated polypeptide comprising an amino acid sequence selected from the group consisting of:
(a) an amino acid sequence according to SEQ ID NO:2;
(b) an amino acid sequence encoded by the canine MC5R clone as deposited with the ATCC and having the ATCC Accession No. PTA-3455;
(c) an extracellular domain of a canine MC5R corresponding to amino acids 1-37, 90-119, 181-185, or 265-272 of SEQ ID NO:2 or of the polypeptide encoded by the canine MC5R clone as deposited with the ATCC and having ATCC Accession No. PTA-3455; and
(d) a cytoplasmic domain of a canine MC5R corresponding to amino acids 63-70, 139-160, 213-237, or 297-325 of SEQ ID NO:2 or of the polypeptide encoded by the canine MC5R clone as deposited with the ATCC and having ATCC Accession No. PTA-3455.

14. An antibody that immuno-specifically binds a polypeptide having the amino acid sequence of SEQ ID NO:2.

15. A method for producing a recombinant canine MC5R polypeptide, comprising:
(a) culturing a host cell transformed with an expression vector which expresses the recombinant canine MC5R polypeptide; and
(b) recovering the recombinant canine MC5R polypeptide from the cell culture.

16. A method for isolating a recombinant canine MC5R polypeptide, comprising:
(a) culturing a host cell transformed with an expression vector which expresses the recombinant canine MC5R polypeptide in the host cell membranes; and
(b) isolating membranes comprising canine MC5R polypeptide from the host cell.

17. A composition comprising a substantially pure polypeptide having the amino acid sequence of SEQ ID NO:2 and a carrier.

18. A method for detecting a polynucleotide comprising the nucleic acid of claim 1 in a sample, comprising:
(a) contacting the sample with a compound that binds to and forms a complex with the polynucleotide for a period sufficient to form the complex; and
(b) detecting the complex,
so that if a complex is detected, the polynucleotide is detected.

19. A method for detecting a polynucleotide comprising the nucleic acid of claim 1 in a sample, comprising:
(a) contacting the sample under stringent hybridization conditions with nucleic acid primers that anneal to the nucleic acid under such conditions; and
(b) amplifying the annealed polynucleotides,
so that if a polynucleotide is amplified, the polynucleotide is detected.

20. The method of claim 19, wherein the polynucleotide is an RNA molecule that encodes a functional MC5R, and the method further comprises reverse transcribing an annealed RNA molecule into a cDNA polynucleotide.

21. A method for identifying a compound that binds to the polypeptide of claim 12, comprising:
(a) contacting a compound with the polypeptide of claim 12 for a time sufficient to form a polypeptide/compound complex; and
(b) determining whether the polypeptide/compound complex is formed,
wherein if the polypeptide/compound complex is formed, then a compound that binds to the polypeptide is identified.

22. A method for identifying a compound that binds to the polypeptide of claim 12 comprising:
(a) contacting a compound with the polypeptide in a cell, for a time sufficient to form a polypeptide/compound complex, wherein the complex drives expression of a reporter gene sequence in said cell; and
(b) detecting the complex by detecting reporter gene sequence expression, so that if a polypeptide/compound complex is detected, a compound that binds to the polypeptide is identified.

23. A method of identifying antagonists of canine MC5R, comprising:
(a) contacting a cell line that expresses a heterologous MC5R polypeptide of claim 12 with a test compound in the presence of an MC5R agonist; and
(b) determining whether the test compound inhibits binding of the MC5R agonist to the MC5R or inhibits functional activity of the MC5R,
in which antagonists are identified as those compounds that inhibit binding of the MC5R agonist to the MC5R or inhibit functional activity of the MC5R.

24. A method for identifying agonists of canine MC5R, comprising:
(a) contacting a cell line that expresses a heterologous polypeptide of claim 12 with a test compound in the presence and in the absence of an MC5R agonist;
(b) determining whether, in the presence of the MC5R agonist, the test compound inhibits the binding of the MC5R agonist to the cell line; and
(c) determining whether, in the absence of the MC5R agonist, the test compound mimics the cellular effects of the MC5R agonist on the cell line,
in which agonists are identified as those test compounds that inhibit the binding but mimic the cellular effects of the MC5R agonist on the cell line.

25. A transgenic animal, the nucleated cells of which comprise a transgene encoding the polypeptide of claim 12.

26. A method for modulating the appetite and/or metabolic rate of an animal in need thereof comprising administering to the animal an effective amount of an MC5R ligand.

27. The method of claim 26 wherein the animal suffers from a decreased-appetite disorder and the MC5R ligand administered is an MC5R antagonist.

28. The method of claim 27, wherein the disorder is anorexia or cachexia.

29. The method of claim 26 wherein the animal suffers from type 2 diabetes or obesity and the MC5R ligand administered is an MC5R agonist.

30. A method of treating skin disorders in an animal comprising administering to an animal in need thereof an effective amount of an MC5R ligand.

31. The method of claim 30 wherein the skin disorder is seborrhea and the MC5R ligand administered is an MC5R antagonist.

32. The method of claim 30 wherein the skin disorder is pruritis or allergic dermatitis and the MC5R ligand administered is an MC5R agonist.

33. A composition comprising an agonist or antagonist of canine MC5R identified by use of the method of claim 23 and a pharmaceutically acceptable carrier.
